(19) United States
(12) Patent Application Publication
(10) Pub. No.: US 2008/0311609 A1
(43) Pub. Date: Dec. 18, 2008

(54) Novel Molecular Probes

(75) Inventors: Barry Greenberg, Sodertalje (SE); Daniel C. Hill, Wilmington, DE (US); Robert Jacobs, Research Triangle Park, NC (US); Sangram S. Sisodia, Chicago, IL (US)

Correspondence Address: Pepper Hamilton LLP, 400 Berwyn Park, 899 Cassatt Road, Berwyn, PA, 19312-1183, US

(73) Assignee: ASTRAZENECA AB, SODERTALJE (SE)

(21) Appl. No.: 11/721,461

(22) PCT Filed: Dec. 14, 2005
(86) PCT No.: PCT/SE05/01920

§ 371 Date: Mar. 26, 2008

Related U.S. Application Data
(60) Provisional application No. 60/635,830, filed on Dec. 14, 2004.

(51) Int. Cl. C12Q 1/02; C07D 267/14; C07D 495/04; C07F 5/02

(52) U.S. Cl. 435/29; 540/491

(57) ABSTRACT

The present invention relates to novel molecular probes having the formula (I) (I) useful for the characterization, detection, localization and isolation of the γ-secretase enzyme.

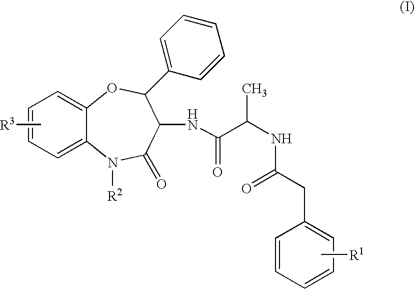

### FIELD OF THE INVENTION

The present invention relates to novel molecular probes useful for the detection, characterization, localization and isolation of the γ-secretase enzyme.

### BACKGROUND OF THE INVENTION

Alzheimer's Disease (AD) is a progressive, neurodegenerative disease characterized clinically by progressive loss of memory, cognition, reasoning, judgment and emotional stability. AD is a common cause of dementia in humans and a leading cause of death in the United States. AD has been observed in races and ethnic groups worldwide and presents a major public health problem throughout the world. No treatment that effectively prevents AD or reverses the clinical symptoms and underlying pathophysiology is currently available and the disease is currently considered among experts to be incurable.

The histopathological manifestations of AD are characteristic lesions known as amyloid (or senile) plaques and neurofibrillar tangles that are found in the regions of the brain associated with memory, reasoning and cognition. Similar alterations are observed in patients with Trisomy 21 (Down's syndrome) and hereditary cerebral hemorrhage with amyloidosis of the Dutch-type.

The major constituent of amyloid plaques is amyloid β protein. Amyloid β protein is derived from the proteolytic cleavage of amyloid precursor protein (APP). Processing of APP to amyloid β protein and other APP fragments is governed by a group of enzymes known as secretases. One type of secretase, γ-secretase, is responsible for the protein cleavage that produces amyloid β protein.

Thus far the nature of the γ-secretase complex has only partly been characterised. It is believed to be a complex of a least four proteins: presenilin 1 (PS1), which mainly occurs as a heterodimer of its N- and C-terminal fragment generated by endoproteolysis of the full length polypeptide, nicastrin, APH-1 and PEN-2. There is currently a need to develop reliable and robust molecular probes useful for studying the biochemical identity of the γ-secretase enzyme and to characterize interactions within the enzyme complex.

Applicants have met this need by developing novel molecular probes useful for elucidating the molecular mechanism of the γ-secretase enzyme.

### DESCRIPTION OF THE INVENTION

Accordingly in a first embodiment of the present invention there is provided a compound of the formula I:

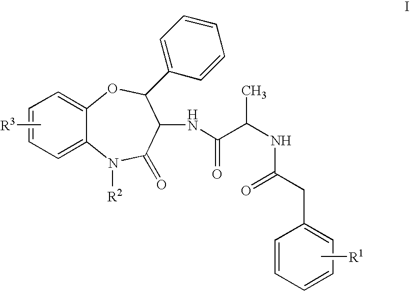

wherein:

R¹ is selected from m-benzoyl, p-benzoyl, or p-azido;

R² is independently selected from H, —CH₃,

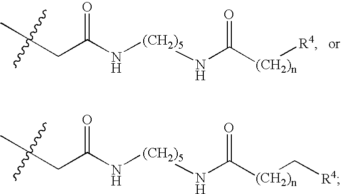

R³ is independently selected from H or

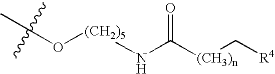

wherein n is 2 or 3; and R⁴ is selected from:

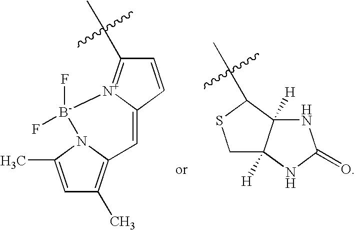

Another embodiment of the invention occurs wherein a compound is selected from:

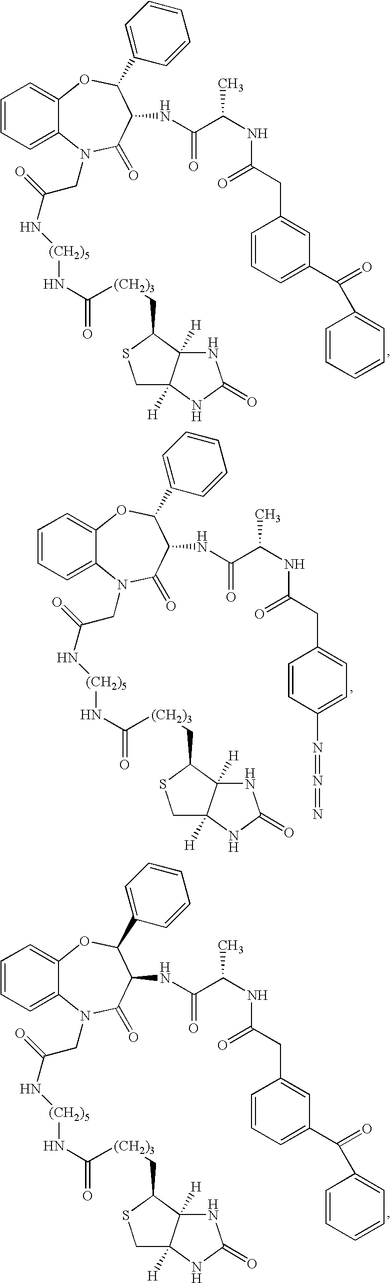

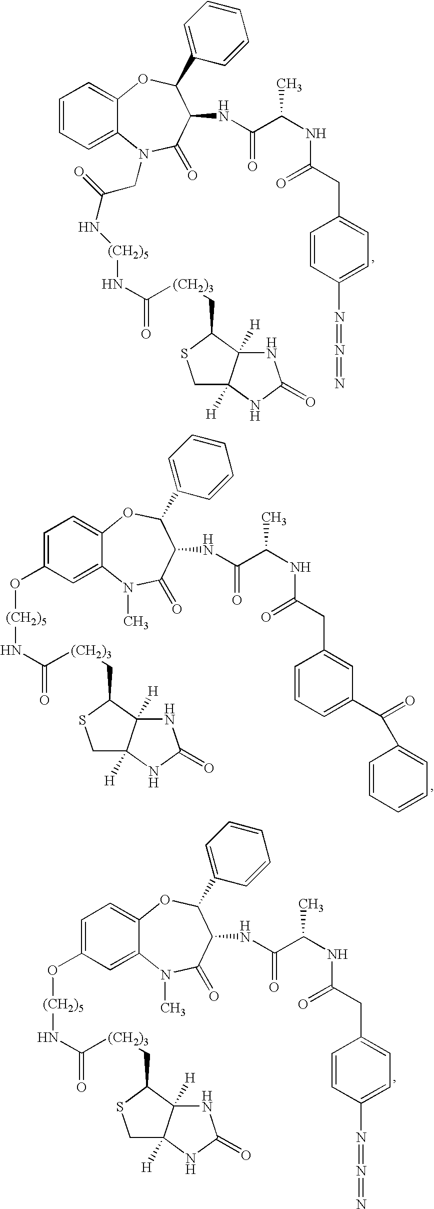

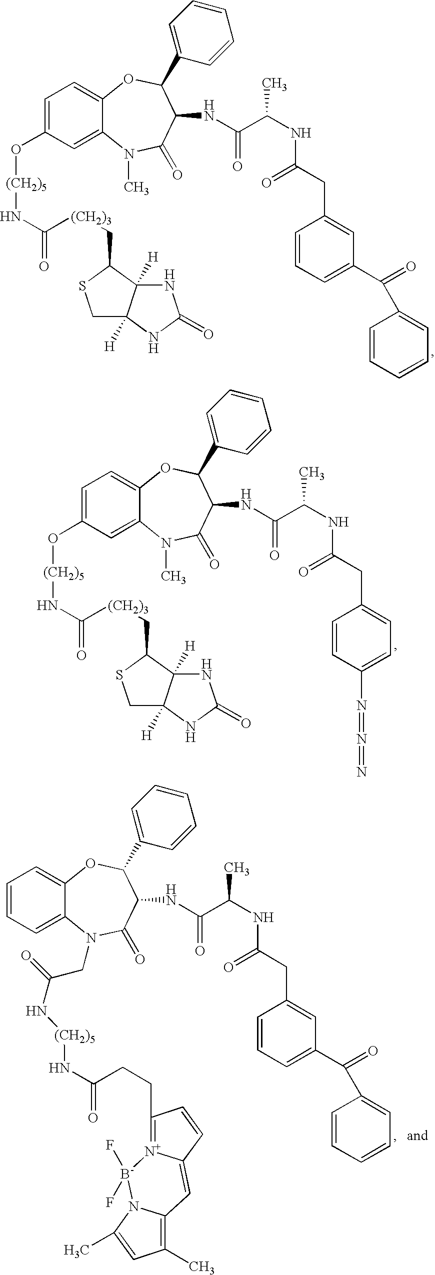

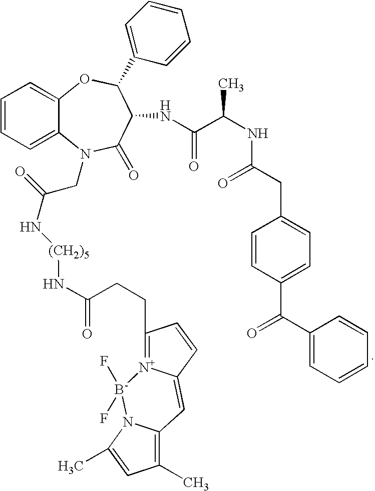

Another embodiment of the invention occurs wherein a compound is selected from the following:
N-[5-({[(2R,3R)-3-({N-[(3-benzoylphenyl)acetyl]-L-alanyl}amino)-2-phenyl-3,4-dihydro-1,5-benzoxazepin-5(2H)-yl]acetyl}amino)pentyl]-4-[(3aS,4S,6aR)-2-oxohexahydro-1H-thieno[3,4-d]imidazol-4-yl]butanamide;
N¹-[(2R,3R)-5-(2-oxo-2-{[5-({4-[(3aS,4S,6aR)-2-oxohexahydro-1H-thieno[3,4-d]imidazol-4-yl]butanoyl}amino)pentyl]amino}ethyl)-2-phenyl-2,3,4,5-tetrahydro-1,5-benzoxazepin-3-yl]-N²-[(4-triaza-1,2-dien-2-ium-1-ylphenyl)acetyl]-L-alaninamide;
N-[5-({[(2S,3S)-3-({N-[(3-benzoylphenyl)acetyl]-L-alanyl}amino)-2-phenyl-3,4-dihydro-1,5-benzoxazepin-5(2H)-yl]acetyl}amino)pentyl]-4-[(3aS,4S,6aR)-2-oxohexahydro-1H-thieno[3,4-d]imidazol-4-yl]butanamide;
N¹-[(2S,3S)-5-(2-oxo-2-{[5-({4-[(3aS,4S,6aR)-2-oxohexahydro-1H-thieno[3,4-d]imidazol-4-yl]butanoyl}amino)pentyl]amino}ethyl)-2-phenyl-2,3,4,5-tetrahydro-1,5-benzoxazepin-3-yl]-N²-[(4-triaza-1,2-dien-2-ium-1-ylphenyl)acetyl]-L-alaninamide;
N-(5-{[(2R,3R)-3-({N-[(3-benzoylphenyl)acetyl]-L-alanyl}amino)-5-methyl-2-phenyl-2,3,4,5-tetrahydro-1,5-benzoxazepin-7-yl]oxy}pentyl)-4-[(3aS,4S,6aR)-2-oxohexahydro-1H-thieno[3,4-d]imidazol-4-yl]butanamide
N¹-((2R,3R)-5-methyl-7-{[5-({4-[(3aS,4S,6aR)-2-oxohexahydro-1H-thieno[3,4-d]imidazol-4-yl]butanoyl}amino)pentyl]oxy}-2-phenyl-2,3,4,5-tetrahydro-1,5-benzoxazepin-3-yl)-N²-[(4-triaza-1,2-dien-2-ium-1-ylphenyl)acetyl]-L-alaninamide
N-(5-{[(2S,3S)-3-({N-[(3-benzoylphenyl)acetyl]-L-alanyl}amino)-5-methyl-2-phenyl-2,3,4,5-tetrahydro-1,5-benzoxazepin-7-yl]oxy}pentyl)-4-[(3aS,4S,6aR)-2-oxohexahydro-1H-thieno[3,4-d]imidazol-4-yl]butanamide
N¹-((2S,3S)-5-methyl-7-{[5-({4-[(3aS,4S,6aR)-2-oxohexahydro-1H-thieno[3,4-d]imidazol-4-yl]butanoyl}amino)pentyl]oxy}-2-phenyl-2,3,4,5-tetrahydro-1,5-benzoxazepin-3-yl)-N²-[(4-triaza-1,2-dien-2-ium-1-ylphenyl)acetyl]-L-alaninamide

The definitions set forth in this section are intended to clarify terms used throughout this application. The term “herein” means the entire application.

When any variable (e.g., R¹, R⁷, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0, 1, 2 or 3 R¹ groups and R^{e} at each occurrence is selected independently from the definition of R^{e}. Also, combinations of substituents and/or variables are permissible only if such combinations result in stable compounds.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Such methods include, but are not limited to, those described below. All references cited herein are hereby incorporated in their entirety by reference.

The novel compounds of this invention may be prepared using the reactions and techniques described in this application. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

The invention is illustrated but not limited by the following examples.

### ABBREVIATIONS

Solvent/Reagent abbreviations:

### DCM Dichloromethane

HATU O-(7-Azabenzotriazole-1-yl)-N,N,N′,N′-tetramethyluronium hexafluorophosphate
PyAOP {7-Azabenzotriazol-1-yloxytris(pyrrolidino)phosphonium hexafluorophosphate

### HOBt Hydroxybenenetriazole

### Et₃N Triethylamine

i-Pr₂EtNDiisopropylethylamine

### NMM N-Methylmorpholine

### MeCN Acetonitrile

### DMF N,N-Dimethylormamide

EtOAc Ethyl acetate
Et₂O Diethyl ether

### THF Tetrahydrofuran

TFA Trifluoroacetic acid

### BOC N-tert-butoxycarbonyl

CBZ carbobenzyloxy
DBU 1,8-diazabicyclo[5.4.0]undec-7-ene

### DIEA N,N-diisopropylethylamine

EDAC-HCl 1-Ethyl-3-(dimethylaminopropyl)carbodiimide hydrochloride
NMM” denotes N-methylmorpholine
p-TSA p-toluenesulfonic acid
TBAB tetrabutylammonium bromide
Ether ethyl ether
Tos-Cl p-toluenesulfonyl chloride
“min.” denotes minutes; “h” denotes hours; “RT” denotes room temperature.

### Experimental Procedures

Unless otherwise noted, organic solutions were “dried” over anhydrous sodium sulfate.

HPLC Method A: Phenomenex Luna 3μ C18(2), 4.6×75 mm column. Solvents: A=H₂O with 0.1% TFA, B=Acetonitrile with 0.1% TFA. Flow rate 2.0 mL/min. 20% B until 0.5 min then a linear gradient to 95% B at 3 min. Maintain at 95% B until 6 min

HPLC Method B: Phenomenex Luna 3μ C18(2), 4.6×75 mm column. Solvents: A=H₂O with 0.1% TFA, B=Acetonitrile with 0.1% TFA. Flow rate 2.0 mL/min. Linear gradient from 10% to 95% B at 5 min. Maintain at 95% B until 7 min.

HPLC Method C: 5μ SB-C8 column 2.1 mm×5 cm. Solvents: A=H₂O with 0.05% TFA, B=10% H₂O, 90% Acetonitrile, 0.05% TFA. Flow rate 1.4 mL/min. Gradient: (5-90% B over 5 min., 90% B hold for 2 min.).

HPLC Method D: Agilent Zorbax 5μ SB-C8 column 2.1 mm×5 cm. Solvents: A=H₂O with 0.1% TFA, B=Acetonitrile with 0.1% TFA. Flow rate 1.4 mL/min. Linear gradient from 9% to 81% B at 3 min. then linear gradient to 95% B at 4 min. Maintain 95% B until 4.5 min.

HPLC Method E: Agilent Zorbax 5μ SB-C8 column 2.1 mm×5 cm. Solvents: A=H₂O with 0.05% TFA, B=90% Acetonitrile, 10% water, 0.05% TFA. Flow rate 1.4 mL/min. Linear gradient from 15% to 90% B in 12 min.

LC/MS:HPLC method: Agilent Zorbax 5μ SB-C8 column 2.1 mm×5 cm. Solvents: A=H₂O with 0.05% TFA, B=10% H₂O, 90% Acetonitrile, 0.05% TFA. Gradient: 10 to 90% B over 3 min., 90% B hold thru 4 min., 10% B at 5 min. and hold at 10% B until 6 min).

Flash column chromatography (fcc=flash column chromatography) was performed using 10 gram packed polypropylene cartridges (Supelco part #57134A) utilizing a step gradient of DCM:MeOH (eluent-start with DCM then add MeOH, 100:1 [symbol] 50:1 [symbol] 20:1) unless otherwise noted.

Preparative Reverse Phase Liquid Chromatography-Research samples were purified* using a Gilson preparative chromatography system. Samples were purified using either a Hewlett Packard CombiHT SB-C18 semi-preparative column (5 μm, 21.2 mm×150 mm; part #870150-902 KJ1018) or a Modcol C18 preparative column (10 μm, 50.8 mm×250 mm; part #PA000-050025). Flow rates; semi-preparative column (20 mL/min), preparative column (50-80 mL/min). Eluent consisted of a mixture of MeCN/H₂O modified w/0.1% TFA.

A typical sequence consisted of:

a) An equilibration (for 3 min at starting gradient concentration)

b) A gradient (started at 40-50% MeCN and ran to 90% MeCN over 7-15 minutes)

c) A flush (for 5 min at 90% MeCN)

*If amines were present products were converted to the free base after purification unless otherwise noted—To remove residual TFA purified products were dissolved in 20% aqueous K₂CO₃ and extracted with DCM. Organic layer was layer was dried over Na₂SO₄, filtered, and solvent was evaporated under reduced pressure. Products were pumped down under high vacuum for 18 h.

LC/MS:HPLC method: Agilent Zorbax 5μ t SB-C8 column 2.1 mm×5 cm. Solvents: A=H₂O with 0.05% TFA, B=10% H₂O, 90% Acetonitrile, 0.05% TFA. Gradient: 10 to 90% B over 3 min., 90% B hold thru 4 min., 10% B at 5 min. and hold at 10% B until 6 min). Molecular ions were M+ or noted otherwise. LC/MS retention times (minutes) are given as part of the characterization data at the end of the experimental section for each compound.

### Molecular Probes

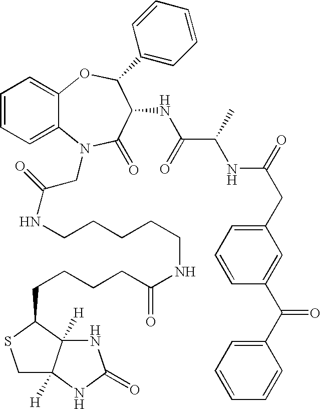

### EXAMPLE 1

### 5-((3aR,6S,6aS)-2-Oxo-hexahydro-thieno[3,4-d]imidazol-6-yl)-pentanoic acid{5-[2-((6R,7S)-7-{(S)-2-[2-(3-benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-amide (1)

To a cooled solution (0° C.) of 5-((3aS,4S,6aR)-2-oxo-hexahydro-thieno[3,4-d]imidazol-4-yl)-pentanoic acid (5-{2-[(6R,7S)-7-((S)-2-amino-propionylamino)-8-oxo-6-phenyl-7,8-dihydro-6H-5-oxa-9-aza-benzocyclohepten-9-yl]-acetylamino}-pentyl)-amide (1h) (66 mg, 0.096 mmol) in DMF (2 mL) under N₂ was added m-benzoylphenylacetic acid (prepared from m-methyl benzophenone using a procedure analogous to that described by J. A. Zderic, J. Org. Chem., 1961, 26, 1635.) (24 mg, 0.100 mmol), HOBt (19 mg, 0.140 mmol), i-Pr₂EtN (40 mL, 0.230 mmol), and EDAC-HCl (27 mg, 0.141 mmol). The mixture was stirred overnight at ambient temperature, diluted with EtOAc (30 mL), then extracted with 20% aqueous K₂CO₃, H₂O (3×20 mL), and brine. The organic solution was dried (Na₂SO₄), filtered and evaporated. The residue was purified by preparative RPHPLC to give 46 mg (52%) of an off-white solid. ¹H NMR (300.132 MHz, MeOH) δ 8.06-7.03 (m, 18H), 5.63 (d, J=7.1 Hz, 1H), 5.21-5.09 (m, 1H), 4.65 and 4.38 (AB, J=16 Hz, 2H), is 4.45 (dd, J=7.7, 4.7 Hz, 1H), 4.26 (dd, J=7.9, 4.4 Hz, 1H), 4.21 (q, J=6.9 Hz, 1H), 3.55 (s, 2H), 3.26-3.04 (m, 5H), 2.89 (dd, J=12.7, 4.9 Hz, 1H), 2.67 (d, J=12.7 Hz, 1H), 2.15 (t, J=7.2 Hz, 2H), 1.81-1.25 (m, 12H), 1.20 (d, J=7.2 Hz, 3H). MS APCI, m/z=916 (M+). LC/MS: 2.33 min. HRMS (TOF ES+) calcd for C₅₀H₅₇N₇O₈S (M+H) 916.4067, found 916.4061.

The starting material (1h) was prepared as follows:

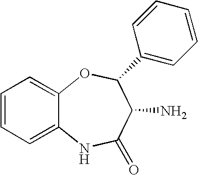

(6R,7S)-7-Amino-6-phenyl-6,7-dihydro-9H-5-oxa-9-aza-benzocyclohepten-8-one hydrochloride (1a) Prepared as described in patent AZ WO2004031154A1.

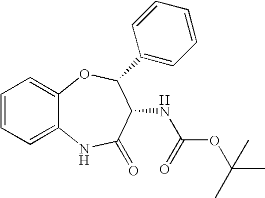

((6R,7S)-8-Oxo-6-phenyl-6,7,8,9-tetrahydro-5-oxa-9-aza-benzocyclohepten-7-yl)-carbamic acid tert-butyl ester (1b) To a slurry of (6R,7S)-7-amino-6-phenyl-6,7-dihydro-9H-5-oxa-9-aza-benzocyclohepten-8-one (1a) (745 mg, 2.56 mmol) in DCM (40 mL) cooled to 0° C. was added Et₃N (720 μL, 5.17 mmol) followed by di-tert-butyl dicarbonate (560 mg, 2.56 mmol). The reaction was stirred at ambient temperature overnight, diluted with 60 mL DCM, extracted with H₂O (2×50 mL), sat'd aqueous NaHCO₃, and brine. The organic layer was dried (Na₂SO₄), filtered, and the solvent removed in vacuo to give a solid (870 mg, 96%). ¹H NMR (300.132 MHz, CDCl3) δ 7.57 (s, 1H), 7.49-7.31 (m, 4H), 7.31-7.11 (m, 4H), 7.09-7.01 (m, 1H), 5.76 (d, J=6.4 Hz, 1H), 5.01 (t, J=7.3 Hz, 1H), 4.94 (d, J=7.7 Hz, 1H), 1.39 (s, 9H); MS ES+, m/z=377 (M+Na⁺). LC/MS: 2.34 min.

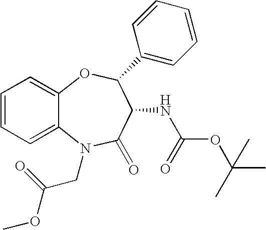

((6R,7S)-7-tert-Butoxycarbonylamino-8-oxo-6-phenyl-7,8-dihydro-6H-5-oxa-9-aza-benzocyclohepten-9-yl)-acetic acid methyl ester (1c) A solution of ((6R,7S)-8-Oxo-6-phenyl-6,7,8,9-tetrahydro-5-oxa-9-aza-benzocyclohepten-7-yl)-carbamic acid tert-butyl ester (1b) (349 mg, 0.985 mmol) in 10 mL DMF was stirred with powdered Cs₂CO₃ for 15 minutes. To this was added methyl bromoacetate (110 μL, 1.16 mmol) and the reaction mixture was stirred for 24 h. At this time, the reaction was diluted with EtOAc (100 mL), extracted with H₂O (4×50 mL), 20% aqueous K₂CO₃, and brine. The organic layer was dried (Na₂SO₄), filtered, and the solvent removed in vacuo. The crude product (clear oil) was purified by fcc on silica (DCM:EtOAc eluent) to give a white foam (383 mg, 91%).

¹H NMR (300.132 MHz, CDCl3) δ 7.50-7.31 (m, 5H), 7.31-7.15 (m, 4H), 5.70 (d, J=7.0 Hz, 1H), 5.02 (t, J=7.4 Hz, 1H), 4.92 (d, J=7.6 Hz, 1H), 4.69 and 4.54 (AB, J=17.2 Hz, 2H), 3.79 (s, 3H), 1.38 (s, 9H); MS ES+, m/z=327 (M+H-100). LC/MS: 2.51 min.

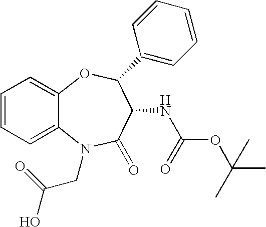

((6R,7S)-7-tert-Butoxycarbonylamino-8-oxo-6-phenyl-7,8-dihydro-6H-5-oxa-9-aza-benzocyclohepten-9-yl)-acetic acid (1d) To a stirred solution of ((6R,7S)-7-tert-butoxycarbonylamino-8-oxo-6-phenyl-7,8-dihydro-6H-5-oxa-9-aza-benzocyclohepten-9-yl)-acetic acid methyl ester (1c) (1.10 g, 2.57 mmol) in THF (27 mL) was added aqueous lithium hydroxide (0.6 M, 9 mL), followed by 3 mL of MeOH. The mixture stirred at RT for 1 h, quenched with 0.5 mL HOAc, THF was evaporated, aqueous residue was acidified with a few drops of 1N HCl, and then extracted with DCM (3×30 mL). The organic phases were dried (Na₂SO₄), filtered, and the solvent removed in vacuo to give a white foam (1.08 g, essentially quantitative), which was used without further purification. ¹NMR (300.132 MHz, CDCl3) δ 9.14 (br s, 1H), 7.52-7.13 (m, 9H), 5.70 (d, J=6.9 Hz, 1H), 5.03 (t, J=7.4 Hz, 1H), 4.94 (d, J=7.8 Hz, 1H), 4.72 (d, J=17.5 Hz, 1H), 4.54 (d, J=17.5 Hz, 1H), 1.41-1.33 (m, 9H); MS APCI, m/z=413 (M+H). LC/MS: 2.24 min.

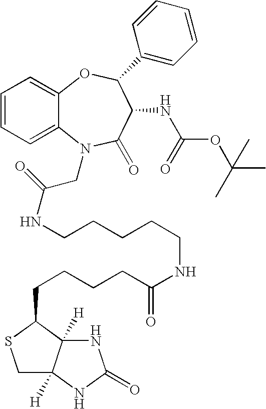

[(6R,7S)-8-Oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-yl]-carbamic acid tert-butyl ester (1e) To a cooled solution (0° C.) of ((6R,7S)-7-tert-butoxycarbonylamino-8-oxo-6-phenyl-7,8-dihydro-6H-5-oxa-9-aza-benzocyclohepten-9-yl)-acetic acid (1d) (445 mg, 1.08 mmol) and 5-((3aR,6S,6aS)-2-oxo-hexahydro-thieno[3,4-d]imidazol-6-yl)-pentanoic acid (5-amino-pentyl)-amide (1j) (355 mg, 1.08 mmol) in DMF (10 mL) under N₂ was added, HOBt (178 mg, 1.32 mmol), N-methylmorpholine (170 μL, 1.56 mmol), and EDAC-HCl (260 mg, 1.36 mmol). The mixture was stirred for 5 h at ambient temperature, diluted with EtOAc (200 mL), extracted with 20% aqueous K₂CO₃, H₂O, and brine. The organic solution was dried (Na₂SO₄), filtered, and evaporated to give solid product which was purified by fcc on silica (198 mg, 25%). ¹H NMR (300.132 MHz, CDCl3) δ 7.50-7.18 (m, 9H), 6.94-6.76 (m, 1H), 6.09-5.98 (m, 1H), 5.96 (s, 1H), 5.67 (d, J=7.2 Hz, 1H), 5.21 (s, 1H), 5.00 (t, J=7.7 Hz, 1H), 4.84 (d, J=7.9 Hz, 1H), 4.62 and 4.48 (AB, J=16.2 Hz, 2H), 4.55-4.41 (m, 1H), 4.35-4.23 (m, 1H), 3.37-3.02 (m, 6H), 2.89 (dd, J=12.8, 4.8 Hz, 1H), 2.70 (d, J=12.7 Hz, 1H), 2.11 (t, J=6.7 Hz, 2H), 1.80-1.55 (m, 6H), 1.53-1.15 (m, 3H), 1.36 (s, 9H); MS APCI, m/z=723 (M+). LC/MS: 2.12 min.

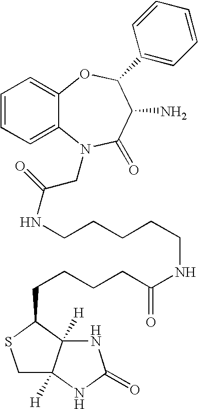

5-((3aR,6S,6aS)-2-Oxo-hexahydro-thieno[3,4-d]imidazol-6-yl)-pentanoic acid{5-[2-((6R,7S)-7-amino-8-oxo-6-phenyl-7,8-dihydro-6H-5-oxa-9-aza-benzocyclohepten-9-yl)-acetylamino]-pentyl}-amide (1f) Using a procedure similar to that described in example (1h), except using [(6R,7S)-8-oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-yl]-carbamic acid tert-butyl ester (1e) (198 mg, 0.274 mmol) as the starting material gave the title compound (1f) (158 mg, 93%) which was used without further purification. ¹H NMR (300.132 MHz, DMSO) δ 7.98 (t, J=5.4 Hz, 1H), 7.69 (t, J=4.9 Hz, 1H), 7.52-7.16 (m, 9H), 6.38 (s, 1H), 6.32 (s, 1H), 5.38 (d, J=6.9 Hz, 1H), 4.57 and 4.25 (AB, J=16.2 Hz, 2H), 4.34-4.24 (m, 1H), 4.17-4.08 (m, 1H), 4.05-3.94 (m, 1H), 3.17-2.91 (m, 5H), 2.81 (dd, J=12.4, 5.0 Hz, 1H), 2.57 (d, J=12.4 Hz, 1H), 2.03 (t, J=7.3 Hz, 2H), 1.70-1.07 (m, 14H); MS APCI, m/z=623 (M+). LC/MS: 1.44 min.

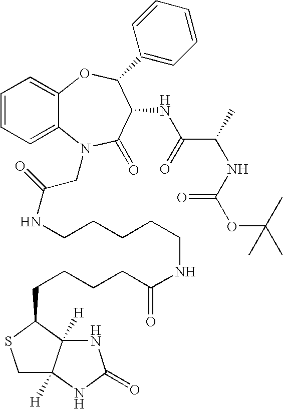

{(S)-1-[(6R,7S)-8-Oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-ylcarbamoyl]-ethyl}-carbamic acid tert-butyl ester (1g) To a cooled solution (0° C.) of 5-((3aR,6S,6aS)-2-oxo-hexahydro-thieno[3,4-d]imidazol-6-yl)-pentanoic acid{5-[2-((6R,7S)-7-amino-8-oxo-6-phenyl-7,8-dihydro-6H-5-oxa-9-aza-benzocyclohepten-9-yl)-acetylamino]-pentyl}-amide (1f) (141 mg, 0.227 mmol) in DMF (5 mL) under N₂ was added N-(tert-butoxycarbonyl)-L-alanine (44 mg, 0.234 mmol), HOBt (40 mg, 0.297 mmol), i-Pr₂EtN (130 μL, 0.746 mmol), and EDAC-HCl (50 mg, 0.258 mmol). The mixture was stirred overnight at ambient temperature, diluted with EtOAc (30 μL), then extracted with 20% aqueous K₂CO₃, H₂O, and brine. The organic solution was dried (Na₂SO₄), filtered and evaporated to give a white solid (138 mg, 77%).

¹H NMR (300.132 MHz, DMSO) δ 8.08 (t, J=5.2 Hz, 1H), 7.70 (t, J=5.4 Hz, 1H), 7.49-7.24 (m, 9H), 7.14 (d, J=5.9 Hz, 1H), 6.96 (d, J=5.4 Hz, 1H), 6.38 (s, 1H), 6.38 (s, 1H), 5.58 (d, J=6.9 Hz, 1H), 4.98 (t, J=6.9 Hz, 1H), 4.56 (d, J=16.4 Hz, 1H), 4.41-4.20 (m, 2H), 4.18-4.04 (m, 1H), 3.85 (t, J=6.8 Hz, 1H), 3.14-2.93 (m, 5H), 2.81 (dd, J=12.4, 5.0 Hz, 1H), 2.57 (d, J=12.4 Hz, 1H), 2.04 (t, J=7.3 Hz, 2H), 1.72-1.13 (m, 11H), 1.32 (s, 9H), 1.06 (d, J=7.2 Hz, 3H); MS APCI, m/z 794 (M+). LC/MS: 1.99 min.

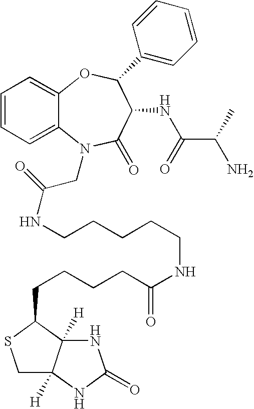

5-((3aS,4S,6aR)-2-Oxo-hexahydro-thieno[3,4-d]imidazol-4-yl)-pentanoic acid (5-{2-[(6R,7S)-7-((S)-2-amino-propionylamino)-8-oxo-6-phenyl-7,8-dihydro-6H-5-oxa-9-aza-benzocyclohepten-9-yl]-acetylamino}-pentyl)-amide (1h) To a solution of {(S)-1-[(6R,7S)-8-oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-ylcarbamoyl]-ethyl}-carbamic acid tert-butyl ester (1g) in 10 mL DCM cooled to 0° C. was added TFA (2 mL). Mixture was warmed to room temperature, stirred for 30 min, solvent/TFA was evaporated, residue was mixed with 20% aqueous K₂CO₃, and extracted with EtOAc:MeOH (25:1, 3×). Extracts were dried (Na₂SO₄), filtered, and evaporated to give a white solid (120 mg, 99%). ¹H NMR (300.132 MHz, DMSO) δ 8.14 (t, J=5.2 Hz, 1H), 7.86-7.58 (m, 2H), 7.51-7.21 (m, 9H), 6.43 (s, 1H), 6.36 (s, 1H), 5.58 (d, J=7.0 Hz, 1H), 5.08-4.83 (m, 1H), 4.61 (d, J=16.4 Hz, 1H), 4.37-4.20 (m, 2H), 4.18-4.06 (m, 2H), 3.25-2.91 (m, 6H), 2.81 (dd, J=12.5, 5.1 Hz, 1H), 2.56 (d, J=12.5 Hz, 1H), 2.04 (t, J=7.2 Hz, 2H), 1.74-1.11 (m, 14H), 1.04 (d, J=6.8 Hz, 3H). MS APCI, m/z=694 (M+). LC/MS: 1.48 min.

### Biotinylated Pentylamine

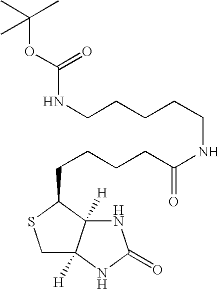

{5-[5-((3aR,6S,6aS)-2-Oxo-hexahydro-thieno[3,4-d]imidazol-6-yl)-pentanoylamino]-pentyl}-carbamic acid tert-butyl ester (1i) Prepared by the method described by Tachibana, K. in Tetrahedron, 2000, 56, 9003.

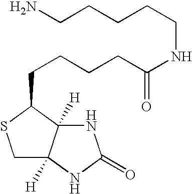

5-((3aR,6S,6aS)-2-Oxo-hexahydro-thieno[3,4-d]imidazol-6-yl)-pentanoic acid (5-amino-pentyl)-amide (1j) To a solution of {5-[5-((3aR,6S,6aS)-2-oxo-hexahydro-thieno[3,4-d]imidazol-6-yl)-pentanoylamino]-pentyl}-carbamic acid tert-butyl ester (1i) (860 mg, 2.00 mmol) in 25 mL DCM was added TFA (4 mL). Mixture was stirred for 2 h, solvent/TFA was evaporated, and residue was pumped under high vac for 1 h. Crude product was dissolved in 30 mL 5:1 DCM:MeOH, 4.65 g (2.34 mmol) Si-amine (Silicycle derivatized silica gel, part #R52030B, loading 1.99 mmol/g) was added, and mixture was stirred for 2 h. Mixture was filtered, silica was washed with DCM, and filtrate was concentrated in vacuo to give a semi-solid. This product was mixed with 10 mL MeCN and left for 1 h, at which time crystallization occurred. Product was collected by filtration and washed with cold MeCN (3×5 mL) to give white solid (583 mg, 89%). ¹H NMR (300.132 MHz, MeOH) δ 7.94 (s, 1H), 4.49 (dd, J=7.9, 4.9 Hz, 1H), 4.30 (dd, J=7.8, 4.5 Hz, 1H), 3.26-3.13 (m, 3H), 2.99-2.86 (m, 3H), 2.70 (d, J=12.7 Hz, 1H), 2.20 (t, J=7.3 Hz, 2H), 1.81-1.32 (m, 12H); MS ES+, m/z=329 (M+H). LC/MS: 0.57 min.

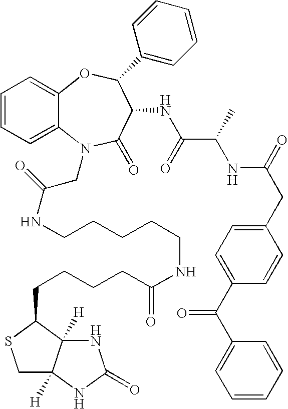

### EXAMPLE 2

### 5-((3aR,6S,6aS)-2-Oxo-hexahydro-thieno[3,4-d]imidazol-6-yl)-pentanoic acid{5-[2-((6R,7S)-7-{(S)-2-[2-(4-benzoyl-phenyl-acetylamino]-propionylamino}-8-oxo-6-phenyl-7,8-dihydro-6H-5-oxa-9-aza-benzocyclohepten-9-yl)-acetylamino]-pentyl}-amide (2)

Using a procedure similar to that described in example (1), except using p-benzoylphenylacetic acid (prepared from p-methyl benzophenone using the procedure described by J. A. Zderic, J. Org. Chem., 1961, 26, 1635.) (24 mg, 0.100 mmol) as the acid component and isolation of title compound by preparative RPHPLC an off-white solid title compound (2 was obtained (48 mg, 53%). ¹H NMR (300.132 MHz, MeOH) δ 7.76 (d, J=7.0 Hz, 2H), 7.72 (d, J=8.2 Hz, 2H), 7.64 (t, J=7.4 Hz, 1H), 7.52 (t, J=7.5 Hz, 2H), 7.47-7.26 (m, 10H), 7.21 (d, J=7.1 Hz, 1H), 5.66 (d, J=7.1 Hz, 1H), 5.27-5.07 (m, 1H), 4.67 (d, J=16.4 Hz, 1H), 4.45 (dd, J=7.8, 5.0 Hz, 1H), 4.38 (d, J=16.4 Hz, 1H), 4.31-4.18 (m, 2H), 3.58 (s, 2H), 3.21 (t, J=6.8 Hz, 2H), 3.17-3.09 (m, 3H), 2.90 (dd, J=12.8, 4.9 Hz, 1H), 2.67 (d, J=12.7 Hz, 1H), 2.16 (t, J=7.2 Hz, 2H), 1.81-1.18 (m, 12H), 1.23 (d, J=7.2 Hz, 3H); MS APCI, m/z=916 (M+). LC/MS: 2.31 min. HRMS (TOF ES+) calcd for C₅₀H₅₇N₇O₈S (M+H) 916.4067, found 916.4069.

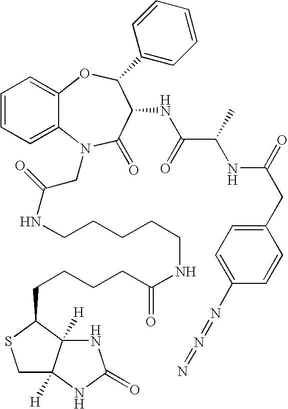

### EXAMPLE 3

### 5-((3aR,6S,6aS)-2-Oxo-hexahydro-thieno[3,4-d]imidazol-6-yl)-pentanoic acid{5-[2-((6R,7S)-7-{(S)-2-[2-(4-azido-phenyl)-acetylamino]-propionylamino}-8-oxo-6-phenyl-7,8-dihydro-6H-5-oxa-9-aza-benzocyclohepten-9-yl)-acetylamino]-pentyl}-amide (3)

Using a procedure similar to that described in example (1), except using p-azidophenylacetic acid prepared from p-amino phenylacetic acid using the procedure described by R. Seyer, J. Med. Chem., 1994, 37, 1841.) (20 mg, 0.113 mmol) as the acid component and isolation of title compound by preparative RPHPLC an off-white solid title compound (a) was obtained (68 mg, 74%). ¹H NMR (300.132 MHz, MeOH) δ 7.47-7.30 (m, 9H), 7.27 (d, J=8.4 Hz, 2H), 6.99 (d, J=8.4 Hz, 2H), 5.65 (d, J=7.1 Hz, 1H), 5.16 (d, J=7.1 Hz, 1H), 4.69 and 4.39 (AB, J=16.4 Hz, 2H), 4.46 (dd, J=7.8, 4.8 Hz, 1H), 4.27 (dd, J=7.9, 4.5 Hz, 1H), 4.20 (q, J=7.2 Hz, 1H), 3.45 (s, 2H), 3.30-3.05 (m, 6H), 2.90 (dd, J=12.7, 5.0 Hz, 1H), 2.68 (d, J=12.7 Hz, 1H), 2.16 (t, J=7.2 Hz, 2H), 1.82-1.26 (m, 14H), 1.20 (d, J=7.2 Hz, 3H); MS APCI, m/z=853 (M+). LC/MS: 2.25 min. HRMS (TOF ES+) calcd for C₄₃H₅₂N₁₀O₇S (M+H) 853.3819, found 853.3823.

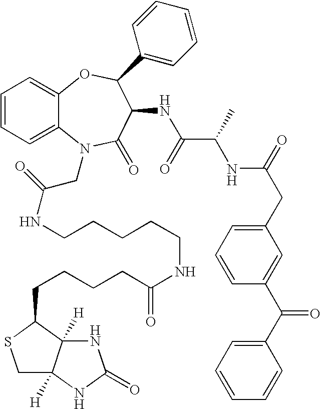

### EXAMPLE 4

### 5-((3aR,6S,6aS)-2-Oxo-hexahydro-thieno[3,4-d]imidazol-6-yl)-pentanoic acid{5-[2-((6S,7R)-7-{(S)-2-[2-(3-benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-amide (4)

To a solution of 5-((3aS,4S,6aR)-2-oxo-hexahydro-thieno[3,4-d]imidazol-4-yl)-pentanoic acid (5-{2-[(6S,7R)-7-((S)-2-amino-propionylamino)-8-oxo-6-phenyl-7,8-dihydro-6H-5-oxa-9-aza-benzocyclohepten-9-yl]-acetylamino}-pentyl)-amide (4k) (76 mg, 0.109 mmol) in 7.5 mL DCM:MeCN (2:1) under N₂ was added m-benzoylphenylacetic acid (25.9 mg, 0.108 mmol), HATU (118 mg, 0.310 mmol), and i-Pr₂EtN (105 μL, 0.603 mmol). The reaction mixture was stirred at ambient temperature overnight. The solvent was evaporated and the residue was purified by preparative RPHPLC to give 54 mg (54%) of an off-white solid. ¹H NMR (300.132 MHz, MeOH) δ 7.83-7.71 (m, 2H), 7.69-7.58 (m, 3H), 7.56-7.26 (m, 13H), 7.11 (d, J=7.1 Hz, 1H), 5.64 (d, J=7.2 Hz, 1H), 5.23-5.15 (m, 1H), 4.68 and 4.36 (AB, J=16.4 Hz, 1H), 4.45 (dd, J=7.6, 4.8 Hz, 1H), 4.31-4.20 (m, 2H), 3.51 (s, 2H), 3.22 (t, J=6.9 Hz, 2H), 3.19-3.14 (m, 1H), 3.13 (t, J=6.8 Hz, 2H), 2.89 (dd, J=12.8, 5.0 Hz, 1H), 2.67 (d, J=12.7 Hz, 1H), 2.15 (t, J=7.2 Hz, 2H), 1.81-1.24 (m, 12H), 1.16 (t, J==3.6 Hz, 3H); MS APCI, m/z=916 (M+). LC/MS: 2.36 min. HRMS (TOF ES+) calcd for C₅₀H₅₇N₇O₈S (M+H) 916.4067, found 916.4093.

The starting material (4k) was prepared as follows:

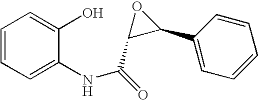

(2R,3S)-3-Phenyl-oxirane-2-carboxylic acid (2-hydroxy-phenyl)-amide (4a) To a stirred suspension of potassium (2R,3S)-3-phenyloxirane-2-carboxylate (prepared as described in patent AZ WO2004031154A1) (6.45 g, 31.9 mmol) in dry THF (100 mL) under nitrogen cooled in an ice-water bath was added isobutyl chloroformate (4.2 mL, 32 mmol) slowly via syringe. NMM (0.80 mL, 7.30 mmol) was added and the mixture stirred while gradually warming to 10° C. over 75 min. The mixture was cooled to 0° C. and 2-aminophenol was added, then the cooling bath was removed and the reaction stirred at ambient temperature for 42 h. The reaction was diluted with Et₂O (100 mL) then filtered through Celite to remove suspended solids. Rotary evaporation of the solution afforded an orange solid that was triturated with diethyl ether (200 mL) and collected by filtration. The solid was rinsed on the filter with additional diethyl ether to afford pure title compound (6.76 g, 83%) as an off-white solid. ¹H NMR (300.132 MHz, DMSO) δ 9.98 (s, 1H), 9.28 (s, 1H), 7.96 (d, J=7.0 Hz, 1H), 7.46-7.31 (m, 5H), 7.01-6.93 (m, 1H), 6.92-6.86 (m, 1H), 6.84-6.75 (m, 1H), 4.22 (d, J=1.6 Hz, 1H), 4.00 (d, J=1.6 Hz, 1H); MS APCI, m/z 256 (M+H). LC/MS: 2.22 min.

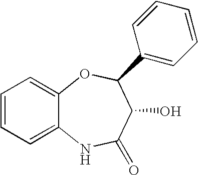

(6S,7S)-7-Hydroxy-6-phenyl-6,7-dihydro-9H-5-oxa-9-aza-benzocyclohepten-8-one (4b) To a solution of (2R,3S)-3-phenyl-oxirane-2-carboxylic acid (2-hydroxy-phenyl)-amide (4a) (5.23 g, 20.5 mmol) in 100 mL MeCN was added Sc(OTf)₃ (0.95 g, 1.92 mmol). Mixture was heated to 66° C. for 75 min, and then cooled to room temperature. Solvent was evaporated under reduced pressure and the residue was purified by fcc on silica (DCM:EtOAc eluent) to give an off-white foam (2.77 g, 53%). ¹H NMR (300.132 MHz, CDCl3) δ 8.30 (s, 1H), 7.51-7.31 (m, 5H), 7.18-7.00 (m, 3H), 6.94-6.82 (m, 1H), 5.29 (d, J=9.8 Hz, 1H), 4.64 (dd, J=9.8, 5.0 Hz, 1H), 3.75 (d, J=5.0 Hz, 1H); MS APCI, m/z=256 (M+H). LC/MS: 1.95 min.

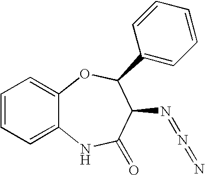

(6S,7R)-7-Azido-6-phenyl-6,7-dihydro-9H-5-oxa-9-aza-benzocyclohepten-8-one (4c) Trifluoromethanesulfonyl chloride (4.5 mL, 42 mmol) was added via syringe to a stirred solution of (6S,7S)-7-hydroxy-6-phenyl-6,7-dihydro-9H-5-oxa-9-aza-benzocyclohepten-8-one (4b) (2.66 g, 10.4 mmol) and Et₃N (5.8 mL, 42 mmol) in DCM (27 mL) under nitrogen at −18° C. The mixture was kept at −18° C. overnight. Additional trifluoromethanesulfonyl chloride (1.2 mL, 11 mmol) and triethylamine (1.5 mL, 11 mmol) was added and the mixture kept at −18° C. for an additional 1 h. The reaction was concentrated in vacuo at −18° C., and the resulting residue immediately dissolved in DMF (25 mL) at −18° C. under nitrogen. Sodium azide (3.86 g, 59.0 mmol) was added to the solution and the mixture warmed to ambient temperature over 30 min. After an additional 2 h, the reaction was diluted with EtOAc (200 mL), extracted with H₂O (4×80 mL), 20% aqueous K₂CO₃ (2×80 mL), and brine. The organic extracts were dried (MgSO₄), filtered and evaporated. The crude product (brown solid) was purified by fcc on silica (DCM:EtOAc eluent) to give the title compound (1.20 g, 41%) as a foamy white solid. ¹H NMR (300.132 MHz, CDCl3) δ 7.67 (br s, 1H), 7.60-7.51 (m, 2H), 7.47-7.37 (m, 3H), 7.29-7.12 (m, 3H), 7.07-6.99 (m, 1H), 5.56 (d, J=6.5 Hz, 1H), 4.46 (d, J=6.0 Hz, 1H); MS APCI, m/z=280 (M+H). LC/MS: 2.14 min.

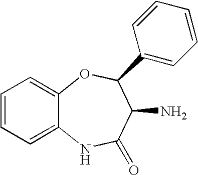

(6S,7R)-7-Amino-6-phenyl-6,7-dihydro-9H-5-oxa-9-aza-benzocyclohepten-8-one hydrochloride (4d) To a Parr® hydrogen flask was added 5% Pd/C (185 mg) followed by 70 mL absolute EtOH. To this was then added (6S,7R)-7-azido-6-phenyl-6,7-dihydro-9H-5-oxa-9-aza-benzocyclohepten-8-one (4c) (1.20 g, 4.28 mmol). Mixture was gently warmed with a heat gun to dissolve azide. Aqueous 1N HCl was added (15 mL), flask was placed on the Parr® shaker, and evacuated/backfilled with hydrogen (4 cycles). Mixture was shaken under 50 psi hydrogen for 2 h (room temperature was 3° C.), filtered through celite, and EtOH was evaporated. Residue was stirred with 250 mL Et₂O for 2 h and precipitated product was filtered, washed (Et₂O), and dried under high vacuum for 18 h. This gave 1.05 g (84%) of a white powder. ¹H NMR (300.132 MHz, DMSO) δ 10.65 (s, 1H), 8.36 (s, 3H), 7.61-7.39 (m, 5H), 7.35-7.14 (m, 4H), 5.88 (d, J=6.1 Hz, 1H), 4.54 (d, J=6.5 Hz, 3H); MS APCI, m/z=255 (M+H). LC/MS: 1.32 min.

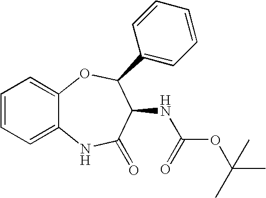

((6S,7R)-8-Oxo-6-phenyl-6,7,8,9-tetrahydro-5-oxa-9-aza-benzocyclohepten-7-yl)-carbamic acid tert-butyl ester (4e) Using a procedure similar to that described in example (1b), except using (6S,7R)-7-amino-6-phenyl-6,7-dihydro-9H-5-oxa-9-aza-benzocyclohepten-8-one hydrochloride (4d) (1.03 g, 3.54 mmol) as the starting material gave the title compound (4e) (1.26 g, 97%) which was used without further purification.

¹H NMR (300.132 MHz, CDCl3) δ 7.94 (s, 1H), 7.49-7.11 (m, 8H), 7.10-6.99 (m, 1H), 5.76 (d, J=6.5 Hz, 1H), 5.09-4.87 (m, 2H), 1.40 (s, 9H); MS APCI, m/z=418 (M+Na⁺+MeCN). LC/MS: 2.56 min.

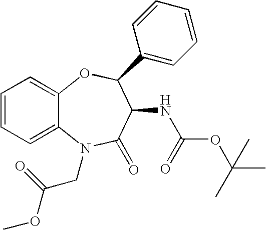

((6S,7R)-7-tert-Butoxycarbonylamino-8-oxo-6-phenyl-7,8-dihydro-6H-5-oxa-9-aza-benzocyclohepten-9-yl)-acetic acid methyl ester (4f) Using a procedure similar to that described in example (1c), except using ((6S,7R)-8-oxo-6-phenyl-6,7,8,9-tetrahydro-5-oxa-9-aza-benzocyclohepten-7-yl)-carbamic acid tert-butyl ester (4e) (1.26 g, 3.54 mmol) as the starting material gave the title compound (4f) (1.46 g, 97%) after purification by fcc.

¹H NMR (300.132 MHz, CDCl3) δ 7.52-7.31 (m, 5H), 7.31-7.15 (m, 4H), 5.70 (d, J=7.0 Hz, 1H), 5.02 (t, J=7.4 Hz, 1H), 4.92 (d, J=7.6 Hz, 1H), 4.68 and 4.54 (AB, J=17.2 Hz, 2H), 3.79 (s, 3H), 1.38 (s, 9H); MS APCI, m/z 449 (M+Na⁺). LC/MS: 2.74 min.

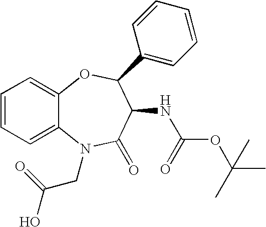

((6S,7R)-7-tert-Butoxycarbonylamino-8-oxo-6-phenyl-7,8-dihydro-6H-5-oxa-9-aza-benzocyclohepten-9-yl)-acetic acid (4g) Using a procedure similar to that described in example (1d), except using ((6S,7R)-7-tert-butoxycarbonylamino-8-oxo-6-phenyl-7,8-dihydro-6H-5-oxa-9-aza-benzocyclohepten-9-yl)-acetic acid methyl ester (4f) (954 mg, 2.24 mmol) as the starting material gave the title compound (4g) (940 mg, essentially quantitative) which was used without further purification. ¹H NMR (300.132 MHz, CDCl3) δ 7.48-7.32 (m, 5H), 7.32-7.18 (m, 4H), 5.70 (d, J=7.0 Hz, 1H), 5.03 (t, J=7.5 Hz, 1H), 4.92 (d, J=7.8 Hz, 1H), 4.73 and 4.56 (AB, J=17.5 Hz, 2H), 1.38 (s, 9H); MS ES+, m/z=413 (M+H). LC/MS: 2.25 min.

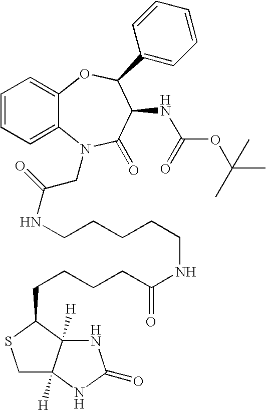

[(6S,7R)-8-Oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-yl]-carbamic acid tert-butyl ester (4h) To a solution of ((6S,7R)-7-tert-butoxycarbonylamino-8-oxo-6-phenyl-7,8-dihydro-6H-5-oxa-9-aza-benzocyclohepten-9-yl)-acetic acid (4g) (385 mg, 0.933 mmol) in 30 mL DCM under N₂ was added 5-((3aR,6S,6aS)-2-oxo-hexahydro-thieno[3,4-d]imidazol-6-yl)-pentanoic acid (5-amino-pentyl)-amide (1j) (308 mg, 0.938 mmol), PyAOP (577 mg, 1.11 mmol), and i-Pr₂EtN (480 μL, 2.76 mmol). The reaction mixture was stirred at ambient for 70 h, the solvent was evaporated, and the residue was dissolved in 200 mL EtOAc:MeOH (20:1). This was extracted with H₂O (3×50 mL), 20% aqueous K₂CO₃, and brine. The solvent was removed in vacuo and the crude product was purified by preparative RPHPLC to give 480 mg (71%) of a white solid. ¹H NMR (300.132 MHz, MeOH) δ 7.52-7.24 (m, 9H), 5.62 (d, J=7.1 Hz, 1H), 4.95 (d, J=7.1 Hz, 1H), 4.67 (d, J=16.4 Hz, 1H), 4.47 (dd, J=7.7, 5.0 Hz, 1H), 4.40 (d, J=16.4 Hz, 1H), 4.28 (dd, J=7.8, 4.4 Hz, 1H), 3.28-3.10 (m, 6H), 2.91 (dd, J=12.8, 4.9 Hz, 1H), 2.69 (d, J=12.8 Hz, 1H), 2.16 (t, J=7.2 Hz, 2H), 1.78-1.27 (m, 12H), 1.37 (s, 9H); MS APCI, m/z=723 (M+). LC/MS: 2.38 min.

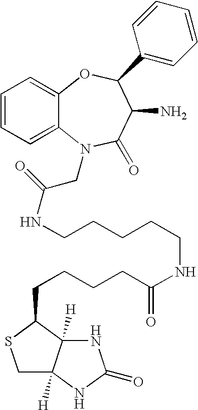

5-((3aR,6S,6aS)-2-Oxo-hexahydro-thieno[3,4-d]imidazol-6-yl)-pentanoic acid{5-[2-((6S,7R)-7-amino-8-oxo-6-phenyl-7,8-dihydro-6H-5-oxa-9-aza-benzocyclohepten-9-yl)-acetylamino]-pentyl}-amide (4i) Using a procedure similar to that described in example (4k), except using [(6S,7R)-8-oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-yl]-carbamic acid tert-butyl ester (4h) (480 mg, 0.664 mmol) as the boc-protected amine component and isolation of title compound (4i) an off-white solid (413 mg, essentially quantitative) was obtained. ¹H NMR (300.132 MHz, MeOH) δ 7.66-7.30 (m, 9H), 5.71 (d, J=7.2 Hz, 1H), 4.80 and 4.44 (AB, J=16.2 Hz, 2H), 4.69 (d, J=7.2 Hz, 1H), 4.51-4.43 (m, 1H), 4.28 (dd, J=7.8, 4.5 Hz, 1H), 3.30-3.09 (m, 6H), 2.92 (dd, J=12.8, 5.0 Hz, 1H), 2.69 (d, J=12.7 Hz, 1H), 2.18 (t, J=7.3 Hz, 2H), 1.79-1.22 (m, 13H)

MS APCI, m/z=623 (M+). LC/MS: 1.72 min.

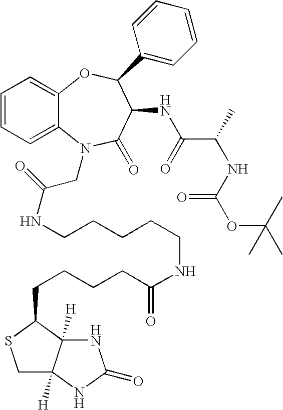

{(S)-1-[(6S,7R)-8-Oxo-9-({5-[5-((3aR,6S,6aS)-2-oxo-hexahydro-tieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-ylcarbamoyl]-ethyl}-carbamic acid tert-butyl ester (4j) To a solution of 5-((3aR,6S,6aS)-2-oxo-hexahydro-thieno[3,4-d]imidazol-6-yl)-pentanoic acid {5-[2-((6S,7R)-7-amino-8-oxo-6-phenyl-7,8-dihydro-6H-5-oxa-9-aza-benzocyclohepten-9-yl)-acetylamino]-pentyl}-amide (4i) (413 mg, 0.664 mmol) in 35 mL DCM under N₂ was added N-(tert-butoxycarbonyl)-L-alanine (133 mg, 0.703 mmol), HATU (382 mg, 1.00 mmol), and i-Pr₂EtN (300 μL, 1.72 mmol). The reaction mixture was stirred at ambient temperature for 7 h, the solvent was evaporated, and the residue was dissolved in 200 mL EtOAc:MeOH (20:1). This was extracted with H₂O (3×50 mL), 20% aqueous K₂CO₃, and brine. The solvent was removed in vacuo and the residue was purified by preparative RPHPLC to give 510 mg (97%) of a white solid. ¹H NMR (300.132 MHz, MeOH) δ 7.53-7.23 (m, 9H), 7.09 (d, J=7.2 Hz, 1H), 5.65 (d, J=7.2 Hz, 1H), 5.27-5.14 (m, 1H), 4.69 and 4.40 (AB, J=16.4 Hz, 2H), 4.47 (dd, J=7.7, 5.0 Hz, 1H), 4.28 (dd, J=−7.8, 4.5 Hz, 1H), 3.99 (q, J=7.1 Hz, 1H), 3.24 (t, J=6.7 Hz, 2H), 3.23-3.16 (m, 1H), 3.15 (t, J=6.9 Hz, 2H), 2.91 (dd, J=12.7, 4.9 Hz, 1H), 2.69 (d, J=12.7 Hz, 1H), 2.17 (t, J=7.2 Hz, 2H), 1.81-1.26 (m, 11H), 1.37 (s, 9H), 1.13 (d, J=7.1 Hz, 3H); MS APCI, m/z=795 (M+H). LC/MS: 2.25 min.

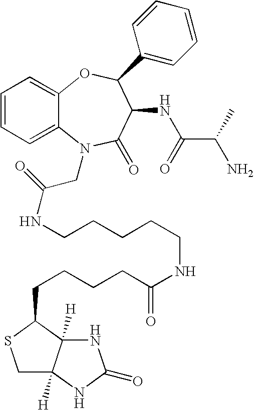

5-((3aS,4S,6aR)-2-Oxo-hexahydro-thieno[3,4-d]imidazol-4-yl)-pentanoic acid (5-{2-[(6S,7R)-7-((S)-2-amino-propionylamino)-8-oxo-6-phenyl-7,8-dihydro-6H-5-oxa-9-aza-benzocyclohepten-9-yl]-acetylamino}-pentyl)-amide (4k) To a solution of {(S)-1-[(6S,7R)-8-oxo-9-({5-[5-((3aR,6S,6aS)-2-oxo-hexahydro-thieno[3,4-d]imidazol-6-yl)-pentanoylamino]-pentylcarbamoyl}-methyl)-6-phenyl-6,7,8,9-tetrahydro-5-oxa-9-aza-benzocyclohepten-7-ylcarbamoyl]-ethyl}-carbamic acid tert-butyl ester (A) (500 mg, 0.630 mmol) in 10 mL DCM was added TFA (4 mL). Mixture was stirred for 2 h, solvent/TFA was evaporated, and residue was pumped under high vac for 1 h. Crude product was dissolved in 30 mL 5:1 DCM:MeOH, 1.92 g (1.30 mmol) Si-amine (Silicycle derivatized silica gel, part #R52030B, loading 1.46 mmol/g) was added, and mixture was stirred for 2 h. Mixture was filtered, silica was washed with DCM, and filtrate was concentrated in vacuo to give a solid (490 mg, essentially quantitative). ¹H NMR (300.132 MHz, MeOH) δ 7.55-7.21 (m, 9H), 5.66 (d, J=7.4 Hz, 1H), 5.27 (d, J=7.3 Hz, 1H), 4.77 and 4.34 (AB, J=16.3 Hz, 2H), 4.47 (dd, J=7.7, 4.9 Hz, 1H), 4.28 (dd, J=7.8, 4.5 Hz, 1H), 3.89 (q, J=6.9 Hz, 1H), 3.30-3.10 (m, 6H), 2.92 (dd, J=12.8, 5.0 Hz, 1H), 2.69 (d, J=12.7 Hz, 1H), 2.17 (t, J=7.2 Hz, 2H), 1.81-1.26 (m, 13H), 1.22 (d, J=7.0 Hz, 3H); MS APCI, m/z=694 (M+). LC/MS: 1.75 min.

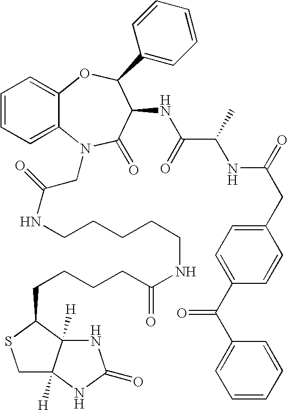

### EXAMPLE 5

### 5-((3aR,6S,6aS)-2-Oxo-hexahydro-thieno[3,4-d]imidazol-6-yl)-pentanoic acid {5-[2-((6S,7R)-7-{(S)-2-[2-(4-benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-amide (5)

Using a procedure similar to that described in example (4), except using p-benzoylphenylacetic acid (26.4 mg, 0.110 mmol) as the acid component and isolation of title compound by preparative RPHPLC an off-white solid title compound (5) was obtained (49 mg, 51%). ¹H NMR (300.132 MHz, MeOH) δ 7.82-7.59 (m, 5H), 7.58-7.48 (m, 2H), 7.45-7.27 (m, 12H), 7.13 (d, J=7.0 Hz, 1H), 5.65 (d, J=7.2 Hz, 1H), 5.26-5.14 (m, 1H), 4.70 and 4.36 (AB, J=16.4 Hz, 2H), 4.45 (dd, J=7.7, 4.3 Hz, 1H), 4.32-4.23 (m, 1H), 3.55 (s, 2H), 3.22 (t, J=6.9 Hz, 2H), 3.13 (t, J=6.9 Hz, 2H), 3.20-3.13 (mH), 2.89 (dd, J=12.8, 5.0 Hz, 1H), 2.67 (d, J=12.7 Hz, 1H), 2.15 (t, J=7.2 Hz, 2H), 1.78-1.25 (m, 13H), 1.19 (d, J=7.2 Hz, 3H); MS APCI, m/z=916 (M+). LC/MS: 2.36 min. HRMS (TOF ES+) calcd for C₅₀H₅₇N₇O₈S (M+H) 916.4067, found 916.4080.

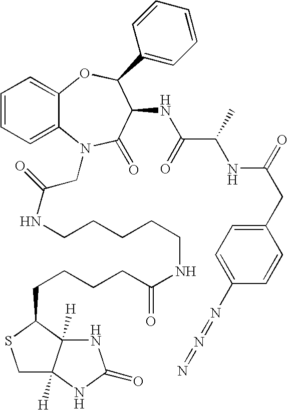

### EXAMPLE 6

### 5-((3aR,6S,6aS)-2-Oxo-hexahydro-thieno[3,4-d]imidazol-6-yl)-pentanoic acid{5-[2-((6S,7R)-7-{(S)-2-[2-(4-azido-phenyl)-acetylamino]-propionylamino}-8-oxo-6-phenyl-7,8-dihydro-6H-5-oxa-9-aza-benzocyclohepten-9-yl)-acetylamino]-pentyl}-amide (6)

Using a procedure similar to that described in example (4), except using p-azidophenylacetic acid (22.0 mg, 0.124 mmol) as the acid component and isolation of title compound by preparative RPHPLC an off-white solid title compound (1 was obtained (49 mg, 51%). ¹H NMR (300.132 MHz, MeOH) δ 7.47-7.27 (m, 10H), 7.21 (d, J=8.5 Hz, 2H), 7.10 (d, J=7.1 Hz, 1H), 6.96 (d, J=8.4 Hz, 2H), 5.64 (d, J=7.2 Hz, 1H), 5.26-5.14 (m, 1H), 4.70 and 4.37 (AB, J=16.4 Hz, 2H), 4.46 (dd, J=7.7, 4.8 Hz, 1H), 4.32-4.21 (m, 2H), 3.41 (s, 1H), 3.24 (t, J=6.9 Hz, 2H), 3.22-3.15 (m, 1H), 3.14 (t, J=6.8 Hz, 2H), 2.90 (dd, J=12.8, 5.0 Hz, 1H), 2.68 (d, J=12.7 Hz, 1H), 2.16 (t, J=7.2 Hz, 2H), 1.80-1.25 (m, 13H), 1.17 (d, J=7.2 Hz, 3H); MS APCI, m/z=853 (M+). LC/MS: 2.28 min. HRMS (TOF ES+) calcd for C₄₃H₅₂N₁₀O₇S (M+H) 853.3819, found 853.3836.

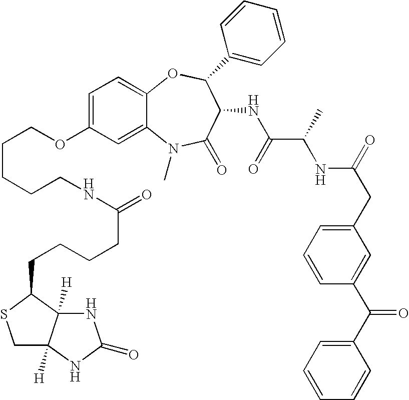

### EXAMPLE 7

### 5-((3aR,6S,6aS)-2-Oxo-hexahydro-thieno[3,4-d]imidazol-6-yl)-pentanoic acid[5-((6R,7S)-7-{(S)-2-[2-(3-benzoyl-phenyl)-acetylamino]-propionylamino}-9-methyl-8-oxo-6-phenyl-6,7,8,9-tetrahydro-5-oxa-9-aza-benzocyclohepten-2-yloxy)-pentyl]-amide (7)

To a solution of 5-((3aR,6S,6aS)-2-oxo-hexahydro-thieno[3,4-d]imidazol-6-yl)-pentanoic acid{5-[(6R,7S)-7-((S)-2-amino-propionylamino)-9-methyl-8-oxo-6-phenyl-6,7,8,9-tetrahydro-5-oxa-9-aza-benzocyclohepten-2-yloxy]-pentyl}-amide (7p) (70.7 mg, 0.106 mmol) in DMF (7 mL) under N₂ was added m-benzoylphenylacetic acid (35.0 mg, 0.146 mmol), HOBt (21.2 mg, 0.157 mmol), i-Pr₂EtN (60 μL, 0.340 mmol), and EDAC-HCl (33.2 mg, 0.173 mmol). The mixture was stirred overnight at ambient temperature, DMF was evaporated, and the residue was purified by preparative RPHPLC to give 52 mg (56%) of a white solid. ¹H NMR (300.132 MHz, MeOH) δ 7.87-7.41 (m, 10H), 7.41-7.23 (m, 5H), 7.17 (d, J=8.8 Hz, 1H), 7.12 (d, J=7.1 Hz, 1H), 6.93 (d, J=2.8 Hz, 1H), 6.85 (dd, J 8.8, 2.8 Hz, 1H), 5.54 (d, J=7.1 Hz, 1H), 5.08-4.98 (m, 1H), 4.43 (dd, J=7.6, 4.6 Hz, 1H), 4.26 (dd, J=7.8, 4.4 Hz, 1H), 4.18 (q, J=7.2 Hz, 1H), 4.02 (t, J=6.2 Hz, 2H), 3.56 (s, 2H), 3.42 (s, 3H), 3.27-3.12 (m, 2H), 2.93-2.79 (m, 1H), 2.65 (d, J=12.7 Hz, 1H), 2.20 (t, J=7.2 Hz, 2H), 1.89-1.35 (m, 12H), 1.20 (d, J=7.2 Hz, 12H); MS APCI, m/z=889 (M+). LC/MS: 2.29 min. HRMS (TOF ES+) calcd for C₄₉H₅₆N₆O₈S (M+H) 889.3953, found 889.3952.

The starting material (7p) was prepared as described below:

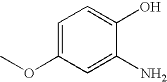

2-Amino-4-methoxy-phenol (7a) Prepared by the method according to Lok, R. in J. Org. Chem., 1996, 61, 3289.

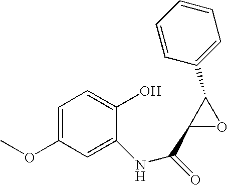

(2R,3S)-3-Phenyl-oxirane-2-carboxylic acid (2-hydroxy-5-methoxy-phenyl)-amide (7b) Using a procedure similar to that described in example (4a), except using potassium (2R,3S)-3-phenyloxirane-2-carboxylate (4.03 g, 19.9 mmol) as the acid component, 2-amino-4-methoxy-phenol as the amine component (2.81 g, 20.2 mmol), and isolation of title compound (7b) a tan solid (4.12 mg, 72%) was obtained. ¹H NMR (300.132 MHz, DMSO) δ 9.51 (s, 1H), 9.25 (s, 1H), 7.68 (d, J=2.9 Hz, 1H), 7.45-7.33 (m, 5H), 6.81 (d, J=8.8 Hz, 1H), 6.56 (dd, J=8.7, 3.0 Hz, 1H), 4.23 (d, J=1.5 Hz, 1H), 4.01 (d, J=1.5 Hz, 1H), 3.67 (s, 3H); MS APCI, m/z=286 (M+H). LC/MS: 2.02 min.

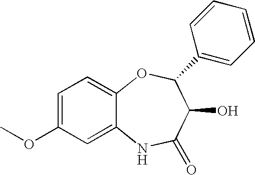

(6R,7R)-7-Hydroxy-2-methoxy-6-phenyl-6,7-dihydro-9H-5-oxa-9-aza-benzocyclohepten-8-one (7c) Using a procedure similar to that described in example (4b), except using (2R,3S)-3-phenyl-oxirane-2-carboxylic acid (2-hydroxy-5-methoxy-phenyl)-amide (7b) (860 mg, 3.02 mmol) as the starting material gave the title compound (7c) (500 mg, 58%). ¹H NMR (300.132 MHz, CDCl3) δ 8.03 (s, 1H), 7.49-7.32 (m, 5H), 6.76 (d, J=8.6 Hz, 1H), 6.66-6.55 (m, 2H), 5.25 (d, J=9.8 Hz, 1H), 4.65 (dd, J=9.8, 5.3 Hz, 1H), 3.78 (s, 3H), 3.70 (d, J=5.3 Hz, 1H); MS APCI, m/z=286 (M+H). LC/MS: 1.77 min.

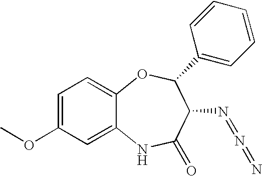

(6R,7S)-7-Azido-2-methoxy-6-phenyl-6,7-dihydro-9H-5-oxa-9-aza-benzocyclohepten-8-one (7d) Using a procedure similar to that described in example (4c), except using (6R,7R)-7-hydroxy-2-methoxy-6-phenyl-6,7-dihydro-9H-5-oxa-9-aza-benzocyclohepten-8-one (7c) (2.49 g, 8.72 mmol) as the starting material gave the title compound (7d) (1.73 g, 64%) after purification by fcc silica (DCM:EtOAc eluent). ¹H NMR (300.132 MHz, CDCl3) δ 8.27 (s, 1H), 7.59-7.50 (m, 2H), 7.44-7.36 (m, 3H), 7.15 (d, J=8.9 Hz, 1H), 6.72 (dd, J=8.8, 2.9 Hz, 1H), 6.59 (d, J=2.9 Hz, 1H), 5.52 (d, J=6.3 Hz, 1H), 4.45 (d, J=6.3 Hz, 1H), 3.77 (s, 3H); MS ES+, m/z=311 (M+H). LC/MS: 2.18 min.

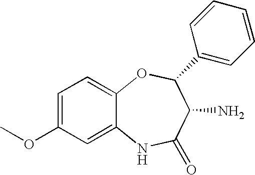

(6R,7S)-7-Amino-2-methoxy-9-methyl-6-phenyl-6,7-dihydro-9H-5-oxa-9-aza-benzocyclohepten-8-one hydrochloride (7e) Using a procedure similar to that described in example (4d), except using (6R,7S)-7-azido-2-methoxy-6-phenyl-6,7-dihydro-9H-5-oxa-9-aza-benzocyclohepten-8-one (7d) (1.19 g, 3.83 mmol) as the starting material gave the title compound (7e) (1.12 g, 91%). The only exception was the Et₂O trituration was not performed. ¹H NMR (300.132 MHz, DMSO) δ 10.60 (s, 1H), 8.34 (br s, 3H), 7.60-7.39 (m, 5H), 7.23 (d, J=8.8 Hz, 1H), 6.82 (dd, J=8.8, 2.9 Hz, 1H), 6.71 (d, J=2.9 Hz, 1H), 5.80 (d, J=6.8 Hz, 1H), 4.50 (d, J=6.8 Hz, 1H), 3.77 (s, 3H); MS APCI, m/z=285 (M+H). LC/MS: 1.26 min.

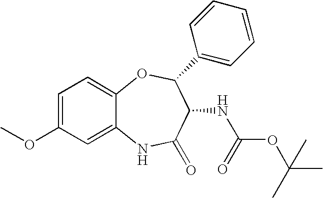

((6R,7S)-2-Methoxy-8-oxo-6-phenyl-6,7,8,9-tetrahydro-5-oxa-9-aza-benzocyclohepten-7-yl)-carbamic acid tert-butyl ester (7f) Using a procedure similar to that described in example (1b), except using (6R,7S)-7-amino-2-methoxy-9-methyl-6-phenyl-6,7-dihydro-9H-5-oxa-9-aza-benzocyclohepten-8-one hydrochloride (7e) (420 mg, 1.29 mmol) as the starting material gave the title compound (7f) (425 mg, 86%) after purification by fcc. ¹H NMR (300.132 MHz, CDCl3) δ 7.54 (s, 1H), 7.48-7.31 (m, 5H), 7.17 (d, J=8.8 Hz, 1H), 6.74 (dd, J=8.8, 2.9 Hz, 1H), 6.59 (d, J=2.9 Hz, 1H), 5.69 (d, J=7.0 Hz, 1H), 5.00 (t, J=7.5 Hz, 1H), 4.88 (d, J=7.9 Hz, 1H), 3.80 (s, 3H), 1.39 (s, 9H); MS ES+, m/z=407 (M+Na⁺). LC/MS: 2.38 min.

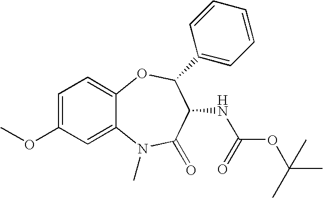

((6R,7S)-2-Methoxy-9-methyl-8-oxo-6-phenyl-6,7,8,9-tetrahydro-5-oxa-9-aza-benzocyclohepten-7-yl)-carbamic acid ter-butyl ester (7g) To a solution of ((6R,7S)-2-methoxy-g-oxo-6-phenyl-6,7,8,9-tetrahydro-5-oxa-9-aza-benzocyclohepten-7-yl)-carbamic acid tert-butyl ester (7f) (425 mg, 1.11 mmol) in 25 mL MeCN was added Cs₂CO₃ (894 mg, 2.74 mmol) and MeI (0.2 mL, 3.2 mmol). Mixture stirred at ambient temperature for 24h. Solvent was evaporated under reduced pressure and the residue was purified by fcc on silica (DCM:EtOAc eluent) to give a white foam (392 mg, 89%). ¹H NMR (300.132 MHz, CDCl3) δ 7.46-7.30 (m, 5H), 7.20-7.11 (m, 1H), 6.84-6.70 (m, 2H), 5.70-5.52 (m, 1H), 5.04-4.85 (m, 2H), 3.83 (s, 3H), 3.46 (s, 3H); MS APCI, m/z=299 (M+H-100). LC/MS: 2.77 min.

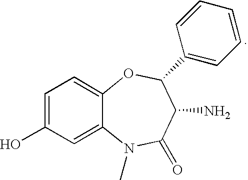

(6R,7S)-7-Amino-2-hydroxy-9-methyl-6-phenyl-6,7-dihydro-9H-5-oxa-9-aza-benzocyclohepten-8-one hydrobromide (7h) To a solution of ((6R,7S)-2-methoxy-9-methyl-8-oxo-6-phenyl-6,7,8,9-tetrahydro-5-oxa-9-aza-benzocyclohepten-7-yl)-carbamic acid tert-butyl ester (2) (580 mg, 1.34 mmol) in DCM (40 mL) was added BBr₃ (2 mL of a 1M solution in DCM). Mixture was stirred at ambient temperature for 1 h, and then additional BBr₃ (2 mL of a 1M solution in DCM) was added. Mixture was stirred for an additional 2 h and then quenched with 2 mL H₂O. DCM/H₂O was evaporated in vacuo and the crude product was purified by fcc on silica to give the title compound (409 mg, 83%) as a tan solid. ¹H NMR (300.132 MHz, DMSO) δ 9.74 (br s, 1H), 8.09 (br s, 3H), 7.52-7.34 (m, 5H), 7.15 (d, J=8.7 Hz, 1H), 6.88 (d, J=2.7 Hz, 1H), 6.73 (dd, J=8.7, 2.7 Hz, 1H), 5.60 (d, J=7.0 Hz, 1H), 4.58 (d, J=6.7 Hz, 1H), 3.41 (s, 3H); MS APCI, m/z=285 (M+H). LC/MS: 1.43 min.

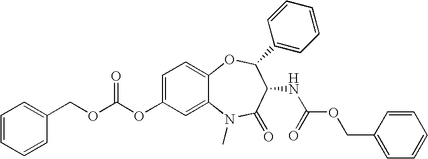

Carbonic acid benzyl ester (6R,7S)-7-benzyloxycarbonylamino-9-methyl-8-oxo-6-phenyl-6,7,8,9-tetrahydro-5-oxa-9-aza-benzocyclohepten-2-yl ester (7i) To a stirred solution of (6R,7S)-7-amino-2-hydroxy-9-methyl-6-phenyl-6,7-dihydro-9H-5-oxa-9-aza-benzocyclohepten-8-one hydrobromide (7h) (404 mg, 1.11 mmol) in 60 mL DCM cooled to (0° C.) was added Et₃N (0.7 mL, 5.0 mmol) followed by dibenzyl dicarbonate (770 mg, 2.69 mmol). Mixture was stirred for 15 min at 0° C., diluted with DCM (100 mL), extracted with H₂O (100 mL), and sat'd aqueous NaHCO₃ (100 mL). The organic extract was dried (Na₂SO₄), filtered, and evaporated. The crude product was purified by fcc on silica (DCM:EtOAc eluent) to give the title compound (419 mg, 68%) as a clear oil. ¹H NMR (300.132 MHz, CDCl3) δ 7.53-7.18 (m, 16H), 7.14-7.03 (m, 2H), 5.65 (d, J=7.0 Hz, 1H), 5.29 (s, 2H), 5.19 (d, J=7.7 Hz, 1H), 5.07 and 4.99 (AB, J=12.2 Hz, 2H), 4.99 (t, J=7.3 Hz, 1H), 3.44 (s, 3H); MS ES+, m/z=553 (M+). LC/MS: 3.08 min.

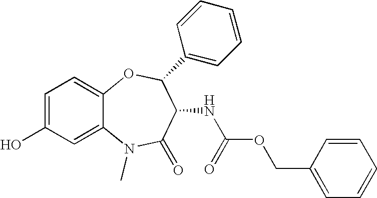

((6R,7S)-2-Hydroxy-9-methyl-8-oxo-6-phenyl-6,7,8,9-tetrahydro-5-oxa-9-aza-benzocyclohepten-7-yl)-carbamic acid benzyl ester (7j) Using a procedure similar to that described in example (1d), except using carbonic acid benzyl ester (6R,7S)-7-benzyloxycarbonylamino-9-methyl-8-oxo-6-phenyl-6,7,8,9-tetrahydro-5-oxa-9-aza-benzocyclohepten-2-yl ester (7i) (417 mg, 0.755 mmol) as the starting material gave the title compound (7j) (316 mg, essentially quantitative) which contained residual benzyl alcohol and was used without further purification. ¹H NMR (300.132 MHz, CDCl3) δ 7.43-7.21 (m, 10H), 7.09 (d, J=8.8 Hz, 1H), 6.70-6.62 (m, 2H), 5.86 (s, 1H), 5.59 (d, J=7.2 Hz, 1H), 5.22 (d, J=8.1 Hz, 1H), 5.08 (d, J=12.3 Hz, 1H), 5.01 (d, J=13.0 Hz, 1H), 4.96 (t, J=7.8 Hz, 1H), 3.42 (s, 3H); MS ES+, m/z=419 (M+). LC/MS: 2.29 min.

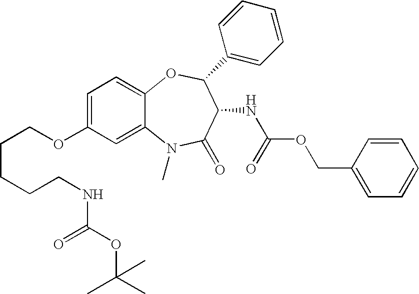

[(6R,7S)-2-(5-tert-Butoxycarbonylamino-pentyloxy)-9-methyl-8-oxo-6-phenyl-6,7,8,9-tetrahydro-5-oxa-9-aza-benzocyclohepten-7-yl]-carbamic acid benzyl ester (7k) To a stirred slurry of K₂CO₃ (721 mg, 5.22 mmol) in DMF (15 mL) was added ((6R,7S)-2-hydroxy-9-methyl-8-oxo-6-phenyl-6,7,8,9-tetrahydro-5-oxa-9-aza-benzocyclohepten-7-yl)-carbamic acid benzyl ester (7j) (315 mg, 0.755 mmol) and (5-bromo-pentyl)-carbamic acid tert-butyl ester (403 mg, 1.51 mmol). Mixture was stirred at ambient temperature for 20 h. Additional K₂CO₃ (584 mg, 4.23 mmol) and (5-bromo-pentyl)-carbamic acid tert-butyl ester (333 mg, 1.25 mmol) was added and mixture was stirred for 5 days. The reaction was diluted with EtOAc (150 mL), extracted with H₂O (4×50 mL), and brine. The organic extracts were dried (Na₂SO₄), filtered, and evaporated. The crude product was purified by fcc on silica (DCM:EtOAc eluent) to give the title compound (429 mg, 94%) as a clear oil.

¹H NMR (300.132 MHz, CDCl3) δ 7.42-7.21 (m, 10H), 7.14 (d, J=9.3 Hz, 1H), 6.80-6.69 (m, 2H), 5.58 (d, J=7.1 Hz, 1H), 5.16 (d, J=7.8 Hz, 1H), 5.07 and 4.99 (AB, J=12.2 Hz, 2H), 5.02-4.94 (m, 1H), 4.53 (br s, 1H), 3.96 (t, J=6.3 Hz, 2H), 3.46 (s, 3H), 3.16 (q, J=6.0 Hz, 2H), 1.83 (quintet, J=6.7 Hz, 2H), 1.64-1.49 (m, 4H), 1.45 (s, 9H); MS ES+, m/z=604 (M+). LC/MS: 2.91 min.

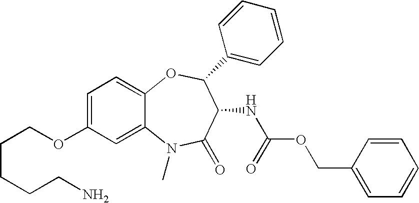

[(6R,7S)-2-(5-Amino-pentyloxy)-9-methyl-8-oxo-6-phenyl-6,7,8,9-tetrahydro-5-oxa-9-aza-benzocyclohepten-7-yl]-carbamic acid benzyl ester (7l) To a solution of [(6R,7S)-2-(5-tert-butoxycarbonylamino-pentyloxy)-9-methyl-8-oxo-6-phenyl-6,7,8,9-tetrahydro-5-oxa-9-aza-benzocyclohepten-7-yl]-carbamic acid benzyl ester (7k) (427 mg, 0.707 mmol) in 10 mL DCM was added TFA (1.2 mL). Mixture was stirred for 2 h, solvent/TFA was evaporated, residue was mixed with 20% aqueous K₂CO₃ (30 mL), and extracted with DCM (3×30 mL). Extracts were dried (Na₂SO₄), filtered, and evaporated to give an oil (357 mg, 99%). ¹H NMR (300.132 MHz, CDCl3) δ 7.41-7.22 (m, 10H), 7.14 (d, J=8.6 Hz, 1H), 6.81-6.69 (m, 2H), 5.54 (d, J=7.1 Hz, 1H), 5.18 (d, J=8.1 Hz, 1H), 5.05 and 4.97 (AB, J=12.2 Hz, 2H), 5.00-4.91 (m, 1H), 3.97 (t, J=5.6 Hz, 2H), 3.44 (s, 3H), 3.05-2.91 (m, 2H), 1.90-1.67 (m, 4H), 1.65-1.49 (m, 2H); MS APCI, m/z=504 (M+). LC/MS: 2.00 min.

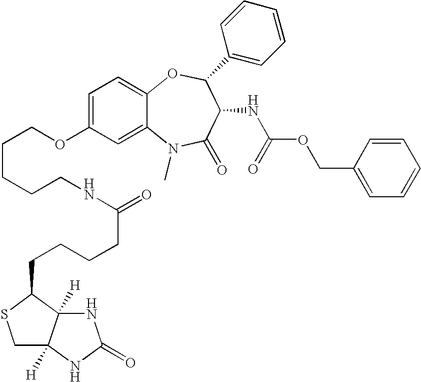

((6R,7S)-9-Methyl-8-oxo-2-{5-[5-((3aR,6S,6aS)-2-oxo-hexahydro-thieno[3,4-d]imidazol-6-yl)-pentanoylamino]-pentyloxy}-6-phenyl-6,7,8,9-tetrahydro-5-oxa-9-aza-benzocyclohepten-7-yl)-carbamic acid benzyl ester (7m) A procedure similar to that described in example (7) was used. The amine component [(6R,7S)-2-(5-amino-pentyloxy)-9-methyl-8-oxo-6-phenyl-6,7,8,9-tetrahydro-5-oxa-9-aza-benzocyclohepten-7-yl]-carbamic acid benzyl ester (7l) (357 mg, 0.707 mmol) was combined with D-biotin (177 mg, 0.725 mmol) to give the title compound as an off-white solid (291 mg, 56%) after preparative RPHPLC. ¹H NMR (300.132 MHz, MeOH) δ 7.41-7.22 (m, 10H), 7.19 (d, J=8.9 Hz, 1H), 6.97 (d, J=2.6 Hz, 1H), 6.86 (dd, J=8.9, 2.9 Hz, 1H), 5.52 (d, J=7.1 Hz, 1H), 5.09 and 4.98 (AB, J=12.7 Hz, 2H), 4.92 (d, J=7.2 Hz, 1H), 4.44 (dd, J=7.7, 4.9 Hz, 1H), 4.27 (dd, J=7.8, 4.4 Hz, 1H), 4.03 (t, J=6.2 Hz, 2H), 3.45 (s, 3H), 3.26-3.13 (m, 3H), 2.88 (dd, J=12.8, 4.3 Hz, 1H), 2.66 (d, J=12.8 Hz, 1H), 2.20 (t, J=7.2 Hz, 2H), 1.82 (quintet, J=8.0 Hz, 2H), 1.76-1.35 (m, 10H); MS APCI, m/z=730 (M+). LC/MS: 2.38 min.

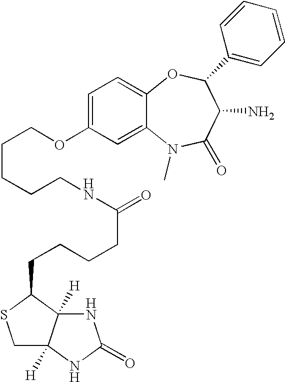

5-((3aR,6S,6aS)-2-Oxo-hexahydro-thieno[3,4-d]imidazol-6-yl)-pentanoic acid[5-((6R,7S)-7-amino-9-methyl-8-oxo-6-phenyl-6,7,8,9-tetrahydro-5-oxa-9-aza-benzocyclohepten-2-yloxy)-pentyl]-amide (7n) To a flask containing ((6R,7S)-9-methyl-8-oxo-2-{5-[5-((3aR,6S,6aS)-2-oxo-hexahydro-thieno[3,4-d]imidazol-6-yl)-pentanoylamino]-pentyloxy}-6-phenyl-6,7,8,9-tetrahydro-5-oxa-9-aza-benzocyclohepten-7-yl)-carbamic acid benzyl ester (ID) (279 mg, 0.382 mmol) was added 10 mL TFA. Mixture was heated to 50° C. for 3 h. TFA was evaporated under reduced pressure, residue was mixed with 20% aqueous K₂CO₃ (50 mL), and extracted with 50:1 DCM:MeOH (3×20 mL). Extracts were dried (Na₂SO₄), filtered, and evaporated under reduced pressure to give a solid (201 mg, 76%). ¹H NMR (300.132 MHz, MeOH) δ 7.51-7.25 (m, 5H), 7.16 (d, J=8.9 Hz, 1H), 6.94 (d, J=2.9 Hz, 1H), 6.83 (dd, J=8.8, 2.8 Hz, 1H), 5.38 (d, J=7.0 Hz, 1H), 4.46 (dd, J=7.1, 4.3 Hz, 1H), 4.28 (dd, J=7.8, 4.4 Hz, 1H), 4.22 (d, J=5.6 Hz, 1H), 4.13-3.96 (m, 3H), 3.45 (s, 3H), 3.27-3.14 (m, 3H), 2.90 (dd, J=12.8, 4.9 Hz, 1H), 2.68 (d, J=12.7 Hz, 1H), 2.20 (t, J=7.2 Hz, 2H), 1.91-1.24 (m, 12H); MS APCI, m/z 596 (M+). LC/MS: 1.63 min.

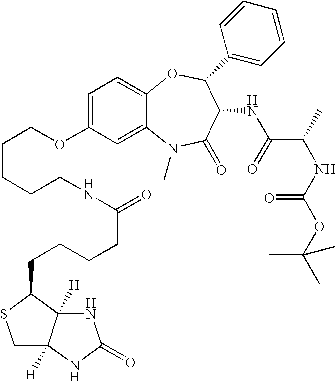

[(S)-1-((6R,7S)-9-Methyl-8-oxo-2-{5-[5-((3aR,6S,6aS)-2-oxo-hexahydro-thieno[3,4-d]imidazol-6-yl)-pentanoylamino]-pentyloxy}-6-phenyl-6,7,8,9-tetrahydro-5-oxa-9-aza-benzocyclohepten-7-ylcarbamoyl)-ethyl]-carbamic acid tert-butyl ester (7o) Using a procedure similar to that described in example (1g), except using 5-((3aR,6S,6aS)-2-oxo-hexahydro-thieno[3,4-d]imidazol-6-yl)-pentanoic acid[5-((6R,7S)-7-amino-9-methyl-8-oxo-6-phenyl-6,7,8,9-tetrahydro-5-oxa-9-aza-benzocyclohepten-2-yloxy)-pentyl]-amide (7n) (228 mg, 0.382 mmol) as the amine component gave the title compound (7o) (163 mg, 55%) after purification by preparative RPHPLC. ¹H NMR (300.132 MHz, MeOH) δ 7.45-7.27 (m, 5H), 7.20 (d, J=8.8 Hz, 1H), 7.12-7.01 (m, 1H), 6.97 (d, J=2.3 Hz, 1H), 6.87 (dd, J=8.7, 2.5 Hz, 1H), 5.59 (d, J=7.1 Hz, 1H), 5.04 (t, J=6.9 Hz, 1H), 4.45 (dd, J=7.5, 4.5 Hz, 1H), 4.28 (dd, J=7.6, 4.3 Hz, 1H), 4.03 (t, J=5.9 Hz, 2H), 3.88 (q, J=7.1 Hz, 1H), 3.46 (s, 3H), 3.26-3.14 (m, 3H), 2.87 (dd, J=12.7, 4.7 Hz, 1H), 2.67 (d, J=12.7 Hz, 1H), 2.20 (t, J=7.0 Hz, 2H), 1.90-1.32 (m, 12H), 1.39 (s, 9H), 1.15 (d, J=7.1 Hz, 3H); MS APCI, m/z=767 (M+). LC/MS: 2.19 min.

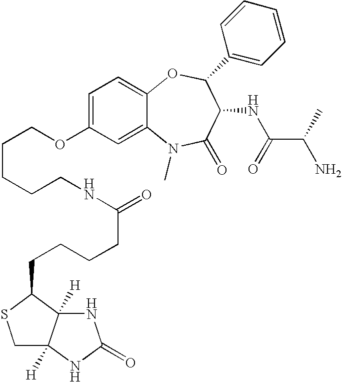

5-((3aR,6S,6aS)-2-Oxo-hexahydro-thieno[3,4-d]imidazol-6-yl)-pentanoic acid{5-[(6R,7S)-7-((S)-2-amino-propionylamino)-9-methyl-8-oxo-6-phenyl-6,7,8,9-tetrahydro-5-oxa-9-aza-benzocyclohepten-2-yloxy]-pentyl}-amide (7p) Using a procedure similar to that described in example (1h), except using [(S)-1-((6R,7S)-9-methyl-8-oxo-2-{5-[5-((3aR,6S,6aS)-2-oxo-hexahydro-thieno[3,4-d]imidazol-6-yl)-pentanoylamino]-pentyloxy}-6-phenyl-6,7,8,9-tetrahydro-5-oxa-9-aza-benzocyclohepten-7-ylcarbamoyl)-ethyl]-carbamic acid tert-butyl ester (7o) (163 mg, 0.213 mmol) as the starting material gave the title compound (7p) (145 mg, essentially quantitative) which was used without further purification. ¹H NMR (300.132 MHz, MeOH) δ 7.46-7.28 (m, 5H), 7.21 (d, J=8.8 Hz, 1H), 6.98 (d, J=2.6 Hz, 1H), 6.87 (dd, J=8.7, 2.7 Hz, 1H), 5.57 (d, J=7.1 Hz, 1H), 5.09 (d, J=7.1 Hz, 1H), 4.46 (dd, J=7.6, 4.4 Hz, 1H), 4.45 (dd, J=7.3, 5.3 Hz, 1H), 4.22 (d, J=5.2 Hz, 1H), 4.09 (q, J=6.2 Hz, 1H), 4.03 (t, J=5.8 Hz, 2H), 3.47 (s, 3H), 3.26-3.14 (m, 2H), 2.89 (dd, J=12.7, 4.9 Hz, 1H), 2.67 (d, J=12.7 Hz, 1H), 2.20 (t, J=6.9 Hz, 2H), 1.90-1.18 (m, 10H), 1.24 (t, J=7.1 Hz, 2H), 1.13 (d, J=6.8 Hz, 3H); MS APCI, m/z=667 (M+). LC/MS: 1.64 min.

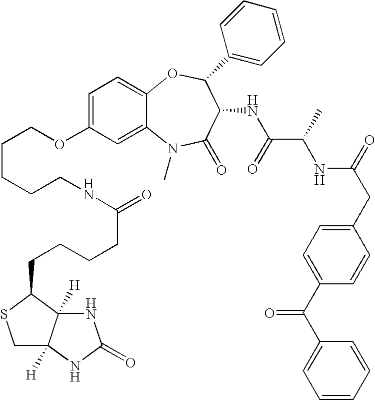

### EXAMPLE 8

### 5-((3aR,6S,6aS)-2-Oxo-hexahydro-thieno[3,4-d]imidazol-6-yl)-pentanoic acid[5-((6R,7S)-7-{(S)-2-[2-(4-benzoyl-phenyl)-acetylamino]-propionylamino}-9-methyl-8-oxo-6-phenyl-6,7,8,9-tetrahydro-5-oxa-9-aza-benzocyclohepten-2-yloxy)-pentyl]-amide (8)

Using a procedure similar to that described in example (7), except using p-benzoylphenylacetic acid (33.5 mg, 0.139 mmol) as the acid component and isolation by preparative RPHPLC a white solid title compound (8) was obtained (67 mg, 71%). ¹H NMR (300.132 MHz, MeOH) δ 7.82-7.58 (m, 4H), 7.52 (t, J=7.4 Hz, 2H), 7.43 (d, J=8.1 Hz, 2H), 7.39-7.25 (m, 4H), 7.19 (d, J=8.8 Hz, 1H), 7.12 (d, J=7.1 Hz, 1H), 6.95 (d, J=2.7 Hz, 1H), 6.86 (dd, J=8.7, 2.7 Hz, 1H), 5.56 (d, J=7.1 Hz, 1H), 5.04 (t, J=7.1 Hz, 1H), 4.43 (dd, J=7.7, 5.0 Hz, 1H), 4.26 (dd, J=7.8, 4.5 Hz, 1H), 4.20 (q, J=7.2 Hz, 1H), 4.02 (t, J=6.2 Hz, 2H), 3.59 (s, 2H), 3.44 (s, 3H), 3.27-3.12 (m, 4H), 2.85 (dd, J=12.7, 5.3 Hz, 1H), 2.64 (d, J=12.7 Hz, 1H), 2.20 (t, J=7.2 Hz, 2H), 1.90-1.35 (m, 12H), 1.22 (d, J=7.2 Hz, 3H); MS APCI, m/z=889 (M+). LC/MS: 2.28 min. HRMS (TOF ES+) calcd for C₄₉H₅₆N₆O₈S (M+H) 889.3953, found 889.3980.

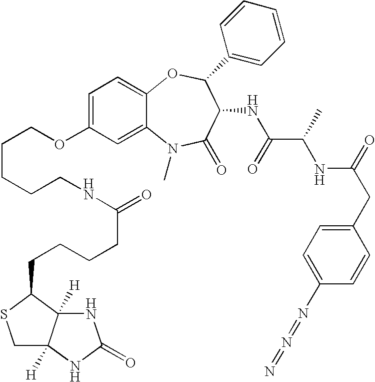

### EXAMPLE 9

### 5-((3aR,6S,6aS)-2-Oxo-hexahydro-thieno[3,4-d]imidazol-6-yl)-pentanoic acid [5-((6R,7S)-7-{(S)-2-[2-(4-azido-phenyl)-acetylamino]-propionylamino}-9-methyl-8-oxo-6-phenyl-6,7,8,9-tetrahydro-5-oxa-9-aza-benzocyclohepten-2-yloxy) pentyl]-amide (9)

Using a procedure similar to that described in example (7), except using p-azidophenylacetic acid (24.2 mg, 0.137 mmol) as the acid component and isolation of title compound by preparative RPHPLC a light-orange solid title compound (2) was obtained (24 mg, 38%). ¹H NMR (300.132 MHz, MeOH) δ 7.42-7.23 (m, 6H), 7.19 (d, J=8.8 Hz, 1H), 7.12 (d, J=7.2 Hz, 1H), 7.00 (d, J=8.5 Hz, 2H), 6.96 (d, J=2.8 Hz, 1H), 6.86 (dd, J=8.8, 2.8 Hz, 1H), 5.56 (d, J=7.2 Hz, 1H), 5.04 (t, J=7.2 Hz, 1H), 4.43 (dd, J=7.7, 4.9 Hz, 1H), 4.27 (dd, J=7.8, 4.4 Hz, 1H), 4.16 (q, J=7.2 Hz, 1H), 4.03 (t, J=6.2 Hz, 2H), 3.46 (s, 3H), 3.27-3.11 (m, 4H), 2.86 (dd, J=12.7, 4.9 Hz, 1H), 2.64 (d, J=12.7 Hz, 1H), 2.20 (t, J=7.2 Hz, 4H), 1.90-1.36 (m, 12H), 1.20 (d, J=7.2 Hz, 3H); MS APCI, m/z=826 (M+). LC/MS: 2.22 min. HRMS (TOF ES+) calcd for C₄₂H₅₁N₉O₇S (M+H) 826.3710, found 826.3748.

### EXAMPLE 10

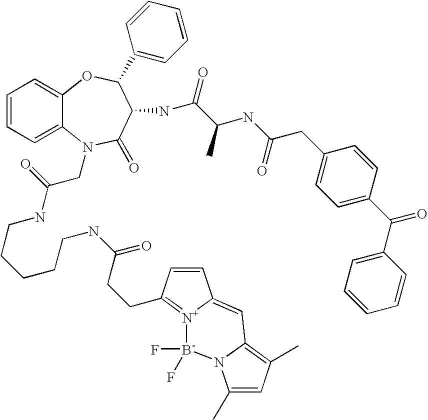

To a solution of (S)—N-{(6R,7S)-9-[(5-amino-pentylcarbamoyl)-methyl]-8-oxo-6-phenyl-6,7,8,9-tetrahydro-5-oxa-9-aza-benzocyclohepten-7-yl}-2-[2-(4-benzoyl-phenyl)-acetylamino]-propionamide (10g) (11 mg, 16 μmol) in DMF (1.5 mL) under N₂ was added 3-(2-{[1-(difluoroboryl)-3,5-dimethyl-1H-pyrrol-2-yl]methylene}-2H-pyrrol-5-yl)propanoic acid (BODIPY®FL) (5.0 mg, 17 μmol), HOBt (6.0 mg, 45 μmol), i-Pr₂EtN (5 μL, 29 μmol), and EDAC-HCl (8.0 mg, 42 μmol). The reaction mixture was stirred at ambient temperature for 3.5 h, the solvent was evaporated, and the residue was purified by preparative RPHPLC to give 9.0 mg (55%) of a bright red solid. ¹H NMR (300.132 MHz, CD3CN) δ 7.84-7.74 (m, 2H), 7.72-7.60 (m, 2H), 7.59-7.26 (m, 1H), 7.00 (d, J=3.8 Hz, 1H), 6.83 (d, J=6.9 Hz, 1H), 6.77-6.66 (m, 1H), 6.62 (d, J=7.0 Hz, 1H), 6.56-6.43 (m, 1H), 6.31 (d, J=3.8 Hz, 1H), 6.23 (s, 1H), 5.64 (d, J=7.1 Hz, 1H), 5.04 (t, J=7.1 Hz, 1H), 4.52 and 4.36 (AB, J=16.3 Hz, 2H), 4.18 (quintet, J=7.1 Hz, 1H), 3.50 and 3.44 (AB, J=14.9 Hz, 2H), 3.27-3.03 (m, 6H), 2.51 (s, 3H), 2.26 (s, 3H), 1.55-1.19 (m, 8H), 1.16 (d, J=7.1 Hz, 3H); MS APCI, m/z=987 (M+Na⁺). LC/MS: 2.83 min. HRMS (TOF ES+) calcd for C₅₄H₅₆N₇O₇BF₂ (M+H) 964.4380, found 964.4369.

The starting material (10g) was prepared as described:

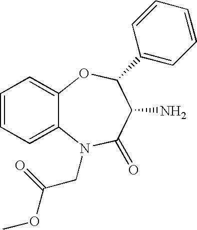

((6R,7S)-7-Amino-8-oxo-6-phenyl-7,8-dihydro-6H-5-oxa-9-aza-benzocyclohepten-9-yl)-acetic acid methyl ester (10a) Using a procedure similar to that described in example (1h), except using ((6R,7S)-7-tert-butoxycarbonylamino-8-oxo-6-phenyl-7,8-dihydro-6H-5-oxa-9-aza-benzocyclohepten-9-yl)-acetic acid methyl ester (1c) (383 mg, 0.898 mmol) as the starting material gave the title compound (10a) (275 mg, 94%) which was used without further purification. ¹H NMR (300.132 MHz, CDCl3) δ 7.55-7.46 (m, 2H), 7.43-7.34 (m, 3H), 7.30-7.19 (m, 4H), 5.48 (d, J=7.1 Hz, 1H), 4.80 and 4.42 (AB, J=17.3 Hz, 2H), 4.17 (d, J=7.1 Hz, 1H), 3.81 (s, 3H), 1.15 (s, 2H); MS APCI, m/z=327 (M+H). LC/MS: 1.54 min.

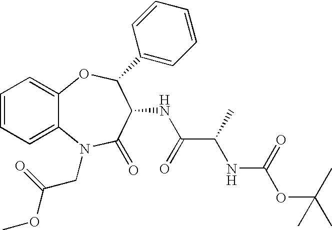

[(6R,7S)-7-((S)-2-tert-Butoxycarbonylamino-propionylamino)-8-oxo-6-phenyl-7,8-dihydro-6H-5-oxa-9-aza-benzocyclohepten-9-yl]-acetic acid methyl ester (10b) Using a procedure similar to that described in example (1g), except using ((6R,7S)-7-amino-8-oxo-6-phenyl-7,8-dihydro-6H-5-oxa-9-aza-benzocyclohepten-9-yl)-acetic acid methyl ester (10a) (270 mg, 0.827 mmol) as the amine component gave the title compound (10b) (412 mg, essentially quantitative) which was used without purification. ¹H NMR (300.132 MHz, CDCl3) δ 7.45-7.34 (m, 5H), 7.32-7.19 (m, 4H), 6.37 (d, J=6.5 Hz, 1H), 5.78 (d, J=7.2 Hz, 1H), 5.18 (t, J=7.1 Hz, 1H), 4.77-4.60 (m, 1H), 4.68 and 4.54 (AB, J=17.2 Hz, 2H), 4.05-3.88 (m, 1H), 3.79 (s, 3H), 1.40 (s, 9H), 1.21 (d, J=7.1 Hz, 3H); MS ES+, m/z=498 (M+H). LC/MS: 2.30 min.

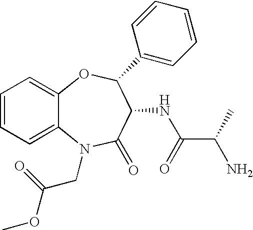

[(6R,7S)-7-((S)-2-Amino-propionylamino)-8-oxo-6-phenyl-7,8-dihydro-6H-5-oxa-9-aza-benzocyclohepten-9-yl]-acetic acid methyl ester (10c) Using a procedure similar to that described in example (7n), except using [(6R,7S)-7-((S)-2-tert-butoxycarbonylamino-propionylamino)-8-oxo-6-phenyl-7,8-dihydro-6H-5-oxa-9-aza-benzocyclohepten-9-yl]-acetic acid methyl ester (10b) (411 mg, 0.827 mmol) as the starting material gave the title compound (10c) (332 mg, essentially quantitative) which was used without further purification. ¹H NMR (300.132 MHz, CDCl3) δ 7.56-7.17 (m, 9H), 5.74 (d, J=7.4 Hz; 1H), 5.22 (t, J=7.4 Hz, 1H), 4.72 and 4.51 (AB, J=17.2 Hz, 2H), 4.47 (s, 3H), 3.33 (q, J=6.8 Hz, 1H), 1.18 (d, J=7.0 Hz, 3H); MS APCI, m/z=398 (M+H). LC/MS: 1.65 min.

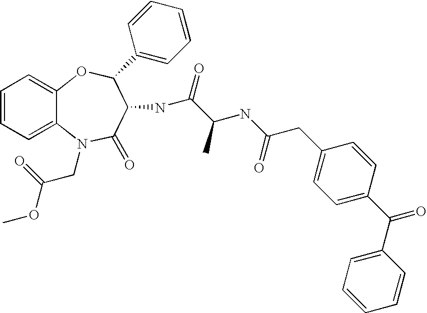

((6R,7S)-7-{(S)-2-[2-(4-Benzoyl-phenyl)-acetylamino]-propionylamino}-8-oxo-6-phenyl-7,8-dihydro-6H-5-oxa-9-aza-benzocyclohepten-9-yl)-acetic acid methyl ester (10d) Using a procedure similar to that described in example (1g), except using [(6R,7S)-7-((S)-2-amino-propionylamino)-8-oxo-6-phenyl-7,8-dihydro-6H-5-oxa-9-aza-benzocyclohepten-9-yl]-acetic acid methyl ester (10c) (166 mg, 0.414 mmol) as the amine component and p-benzoylphenylacetic acid (108 mg, 0.450 mmol) as the acid component in DCM gave the title compound (10d) (233 mg, 91%) after purification by fcc on silica.

¹H NMR (300.132 MHz, CDCl3) δ 7.89-7.72 (m, 4H), 7.66-7.18 (m, 14H), 6.17 (d, J=6.8 Hz, 1H), 5.85 (d, J=6.8 Hz, 1H), 5.72 (d, J=7.1 Hz, 1H), 5.17 (t, J=7.2 Hz, 1H), 4.67 and 4.55 (AB, J=17.3 Hz, 2H), 4.33-4.19 (m, 1H), 3.79 (s, 3H), 3.59 (s, 2H), 1.21 (d, J=7.0 Hz, 3H); MS APCI, m/z=620 (M+). LC/MS: 2.39 min.

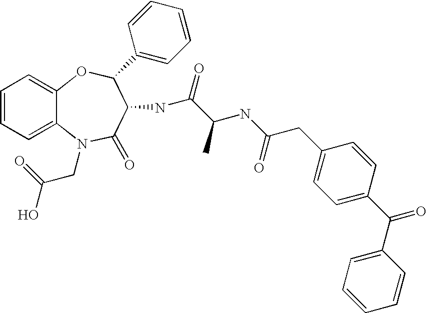

((6R,7S)-7-{(S)-2-[2-(4-Benzoyl-phenyl)-acetylamino]-propionylamino}-8-oxo-6-phenyl-7,8-dihydro-6H-5-oxa-9-aza-benzocyclohepten-9-yl)-acetic acid (10e) Using a procedure similar to that described in example (1d), except using ((6R,7S)-7-{(S)-2-[2-(4-benzoyl-phenyl)-acetylamino]-propionylamino}-8-oxo-6-phenyl-7,8-dihydro-6H-5-oxa-9-aza-benzocyclohepten-9-yl)-acetic acid methyl ester d) (233 mg, 0.376 mmol) as the starting material gave the title compound (10e) (224 mg, 98%) which was used without further purification. ¹H NMR (300.132 MHz, CDCl3) δ 7.89-7.71 (m, 4H), 7.68-7.12 (m, 14H), 6.70 (br s, 1H), 5.60 (d, J=6.9 Hz, 1H), 5.30-5.07 (m, 1H), 4.49 and 3.96 (AB, J=17.1 Hz, 2H), 4.19-3.85 (m, 2H), 3.67 (s, 2H), 0.92 (d, J=6.6 Hz, 3H); MS APCI, m/z=606 (M+). LC/MS: 2.42 min.

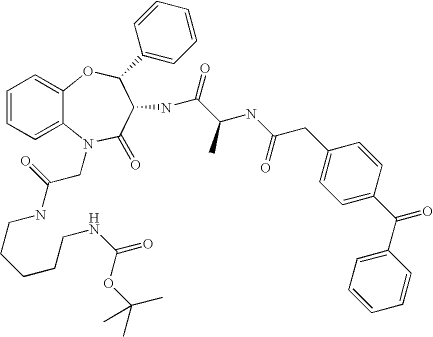

{5-[2-((6R,7S)-7-{(S)-2-[2-(4-Benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-carbamic acid tert-butyl ester (10f) Using a procedure similar to that described in example (1g), except using (5-amino-pentyl)-carbamic acid tert-butyl ester (95 mg, 0.47 mmol) as the amine component and ((6R,7S)-7-{(S)-2-[2-(4-benzoyl-phenyl)-acetylamino]-propionylamino}-8-oxo-6-phenyl-7,8-dihydro-6H-5-oxa-9-aza-benzocyclohepten-9-yl)-acetic acid (10e) (220 mg, 0.360 mmol) as the acid component gave the title compound (10f) (191 mg, 67%) after purification by fcc on silica. ¹H NMR (300.132 MHz, CDCl3) δ 7.85-7.72 (m, 4H), 7.60 (t, J=7.2 Hz, 1H), 7.49 (t, J=7.5 Hz, 2H), 7.44-7.23 (m, 1H), 6.68-6.38 (m, 1H), 6.22 (br s, 0.5H), 5.99 (br s, 0.5H), 5.70 (d, J=7.4 Hz, 1H), 5.13 (t, J=7.3 Hz, 1H), 4.70-4.11 (m, 3H), 3.73-2.82 (m, 6H), 1.56-1.11 (m, 9H), 1.42 (s, 9H); MS ES+, m/z=790 (M+). LC/MS: 2.51 min.

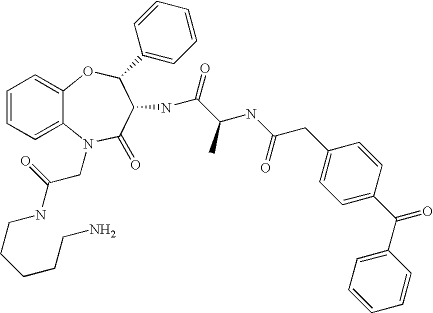

(S)—N-{(6R,7S)-9-[(5-Amino-pentylcarbamoyl)-methyl]-8-oxo-6-phenyl-6,7,8,9-tetrahydro-5-oxa-9-aza-benzocyclohepten-7-yl}-2-[2-(4-benzoyl-phenyl)-acetylamino]-propionamide (10g) Using a procedure similar to that described in example (1h), except using {5-[2-((6R,7S)-7-{(S)-2-[2-(4-benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-carbamic acid tert-butyl ester (10f) (192 mg, 0.242 mmol) as the starting material gave the title compound ((168 mg, essentially quantitative) which was used without further purification. ¹HNMR (300.132 MHz, MeOH) δ 7.82-7.59 (m, 5H), 7.52 (t, J=7.5 Hz, 2H), 7.47-7.25 (m, 11H), 5.66 (d, J=7.1 Hz, 1H), 5.17 (d, J=7.2 Hz, 1H), 4.67 and 4.40 (AB, J=16.4 Hz, 2H), 4.23 (q, J=7.1 Hz, 1H), 3.58 (s, 2H), 3.28-3.13 (m, 2H), 2.68 (t, J=7.0 Hz, 2H), 1.62-1.42 (m, 4H), 1.41-1.15 (m, 5H); MS APCI, m/z=690 (M+). LC/MS: 2.09 min.

### EXAMPLE 11

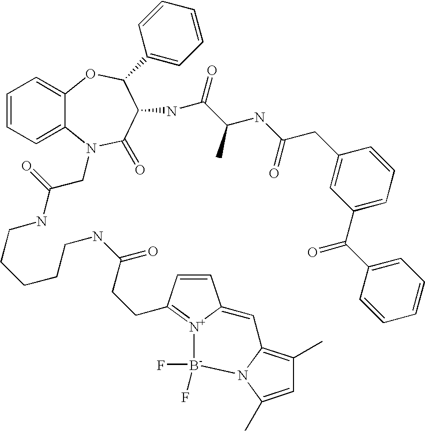

Using a procedure similar to that described in example (10), except using (S)—N-{(6R,7S)-9-[(5-amino-pentylcarbamoyl)-methyl]-8-oxo-6-phenyl-6,7,8,9-tetrahydro-5-oxa-9-aza-benzocyclohepten-7-yl}-2-[2-(3-benzoyl-phenyl)-acetylamino]-propionamide (11d) (27 mg, 39 μmol) as the amine component and isolation of title compound by preparative RPHPLC a bright-red solid title compound (11) was obtained (13 mg, 40%, purity ˜83%).

¹H NMR (300.132 MHz, MeOH) δ 7.81-7.58 (m, 5H), 7.51 (t, J=7.4 Hz, 2H), 7.45-7.26 (m, 11H), 7.19 (d, J=7.1 Hz, 1H), 6.98 (d, J=3.7 Hz, 1H), 6.29 (d, J=4.1 Hz, 1H), 6.18 (s, 1H), 5.66 (d, J=7.0 Hz, 1H), 5.16 (t, J=7.1 Hz, 1H), 4.66 and 4.37 (AB, J=16.4 Hz, 2H), 4.22 (q, J=7.4 Hz, 1H), 3.57 (s, 2H), 3.24-3.09 (m, 6H), 2.49 (s, 3H), 2.25 (s, 3H), 1.57-1.27 (m, 8H), 1.22 (d, J=7.2 Hz, 3H); MS APCI, m/z=987 (M+Na⁺). LC/MS: 2.82 min. HRMS (TOF ES+) calcd for C₅₄H₅₆N₇O₇BF₂ (M+H) 964.4380, found 964.4376.

The starting material (11d) was prepared as described:

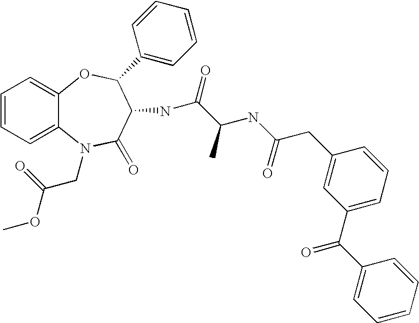

((6R,7S)-7-{(S)-2-[2-(3-Benzoyl-phenyl)-acetylamino]-propionylamino}-X-oxo-6-phenyl-7,8-dihydro-6H-5-oxa-9-aza-benzocyclohepten-9-yl)-acetic acid methyl ester (11a) Using a procedure similar to that described in example (1g), except using [(6R,7S)-7-((S)-2-amino-propionylamino)-8-oxo-6-phenyl-7,8-dihydro-6H-5-oxa-9-aza-benzocyclohepten-9-yl]-acetic acid methyl ester (10c) (166 mg, 0.414 mmol) as the amine component and m-benzoylphenylacetic acid (103 mg, 0.429 mmol) as the acid component in DCM gave the title compound (11a) (245 mg, 95%) after purification by fcc on silica.

¹H NMR (300.132 MHz, CDCl3) δ 7.87-7.66 (m, 4H), 7.63-7.18 (m, 14H), 6.16 (d, J=7.1 Hz, 1H), 5.85 (d, J=6.9 Hz, 1H), 5.71 (d, J=7.1 Hz, 1H), 5.17 (t, J=7.2 Hz, 1H), 4.67 and 4.54 (AB, J=17.3 Hz, 2H), 4.24 (quintet, J=7.0 Hz, 1H), 3.80 (s, 3H), 3.56 (s, 2H), 1.20 (d, J=6.9 Hz, 3H); MS APCI, m/z=620 (M+). LC/MS: 2.39 min.

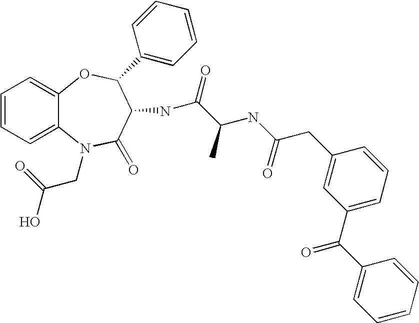

((6R,7S)-7-{(S)-2-[2-(3-Benzoyl-phenyl)-acetylamino]-propionylamino}-8-oxo-6-phenyl-7,8-dihydro-6H-5-oxa-9-aza-benzocyclohepten-9-yl)-acetic acid (11b) Using a procedure similar to that described in example (1d), except using ((6R,7S)-7-{(S)-2-[2-(3-benzoyl-phenyl)-acetylamino]-propionylamino}-8-oxo-6-phenyl-7,8-dihydro-6H-5-oxa-9-aza-benzocyclohepten-9-yl)-acetic acid methyl ester (11a) (245 mg, 0.395 mmol) as the starting material gave the title compound (11b) (234 mg, 97%) which was used without further purification. ¹H NMR (300.132 MHz, CDCl3) δ 7.90-7.38 (m, 15H), 7.37-7.11 (m, 3H), 6.70 (d, J=6.8 Hz, 1H), 5.57 (d, J=7.5 Hz, 1H), 5.23 (t, J=7.9 Hz, 1H), 4.45 and 3.93 (d, J=17.1 Hz, 2H), 4.08-3.93 (m, 1H), 3.81-3.62 (m, 1H), 3.67 (s, 2H), 0.94 (d, J=6.8 Hz, 3H); MS APCI, m/z=606 (M+). LC/MS: 2.42 min.

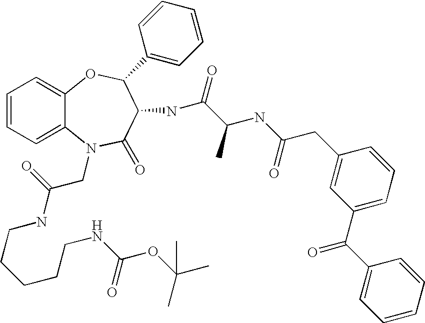

{5-[2-((6R,7S)-7-{(S)-2-[2-(3-Benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-carbamic acid tert-butyl ester (11c) Using a procedure similar to that described in example (1g), except using (5-amino-pentyl)-carbamic acid tert-butyl ester (95 mg, 0.47 mmol) as the amine component and ((6R,7S)-7-{(S)-2-[2-(3-benzoyl-phenyl)-acetylamino]-propionylamino}-8-oxo-6-phenyl-7,8-dihydro-6H-5-oxa-9-aza-benzocyclohepten-9-yl)-acetic acid (11b) (230 mg, 0.380 mmol) as the acid component gave the title compound (11c) (231 mg, 77%) after purification by fcc on silica. ¹H NMR (300.132 MHz, CDCl3) δ 7.80 (d, J=7.3 Hz, 2H), 7.73-7.55 (m, 3H), 7.51-7.21 (m, 13H), 6.62-6.31 (m, 1H), 6.25 (br s, 0.5H), 5.95 (br s, 0.5H), 5.69 (d, J=7.5 Hz, 1H), 5.20-5.06 (m, 1H), 4.68-4.11 (m, 3H), 3.54 (s, 2H), 3.39-3.11 (m, 2H), 2.99 (q, J=6.1 Hz, 2H), 1.57-1.14 (m, 6H), 1.42 (s, 9H), 1.18 (d, J=6.9 Hz, 3H); MS ES+, m/z=790 (M+). LC/MS: 2.52 min.

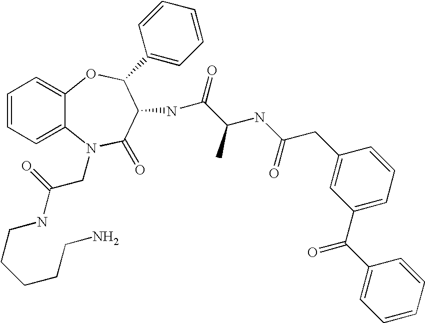

### EXAMPLE 11

### (S)—N-{(6R,7S)-9-[(5-Amino-pentylcarbamoyl)-methyl]-8-oxo-6-phenyl-6,7,8,9-tetrahydro-5-oxa-9-aza-benzocyclohepten-7-yl}-2-[2-(3-benzoyl-phenyl)-acetylamino]-propionamide (11d)

Using a procedure similar to that described in example (1h), except using {5-[2-((6R,7S)-7-{(S)-2-[2-(3-benzoyl-phenyl)-acetylamino]-propionylamino}-8-oxo-6-phenyl-7,8-dihydro-6H-5-oxa-9-aza-benzocyclohepten-9-yl)-acetylamino]-pentyl}-carbamic acid tert-butyl ester (11c) (231 mg, 0.292 mmol) as the starting material gave the title compound (11d) (211 mg, essentially quantitative) which was used without further purification. ¹H NMR (300.132 MHz, MeOH) δ 7.83-7.73 (m, 2H), 7.72-7.59 (m, 3H), 7.58-7.23 (m, 13H), 5.63 (d, J=7.2 Hz, 1H), 5.14 (d, J=7.1 Hz, 1H), 4.64 and 4.40 (AB, J=16.4 Hz, 2H), 4.21 (q, J=7.2 Hz, 1H), 3.54 (s, 2H), 3.28-3.16 (m, 2H), 2.66 (t, J=6.8 Hz, 2H), 1.60-1.42 (m, 4H), 1.40-1.26 (m, 2H), 1.20 (d, J=7.3 Hz, 3H); MS APCI, m/z=690 (M+). LC/MS: 2.10 min.

### EXAMPLE 12

### Gamma Secretase Detergent Extract Assay

The gamma secretase enzyme assay measures the amount of amyloid β (Aβ)40 product generated by the cleavage of C100, a truncated form of amyloid precursor protein (APP). The C100 substrate is a recombinant protein purified from E. coli inclusion bodies. The γsecretase enzyme complex is prepared by detergent extraction of HeLa 8A8 cell membranes. The enzyme reaction contains 10 ul of inhibitor at a defined concentration, diluted from a DMSO stock into 96-well microplates (final concentration of DMSO is maintained at 5%). 20 ul of the C100 substrate (600 nM final concentration), in reaction buffer, (50 mM MES, pH 6.5, containing 100 mM NaCl, 1 mM EDTA, 1 mM DTT, 1 mg/mL BSA, 0.25% Chapso, 0.01% PE, 0.01% PC and a protease cocktail), is added to the plates. The reactions are initiated by addition of 10 ul enzyme at a 20-fold dilution from stock. An Aβ40 standard curve diluted in the reaction buffer plus C 100 is included in each assay. Plates are incubated for 3 hours at 37 degrees. After the incubation period, 50 ul of an antibody mixture is added: rabbit anti-Aβ40 antibody (Biosource #44-3481) at 0.16 ug/ml and biotinylated 4G8 (Senetek #240-10) at 0.25 ug/ml in DPBS (Fisher #MT21031CV) containing 0.5% bovine serum albumin, 0.5% Tween 20. Plates are then incubated overnight at 4 degrees. The following morning, a 50 ul mixture of 0.0625 mg/ml Ruthenium labeled goat anti-rabbit IgG (labeled in-house) and 125 ug/ml of Streptavadin beads (Igen #M280), diluted in the same DPBS buffer, is added to detect the cleaved product. After a one hour incubation period at room temperature, an Igen M Series instrument is utilized to quantitate the results by electrochemiluminescence.

### EXAMPLE 13

### Gamma Secretase Whole Cell Assay (GSWC)

Preparation of cells for assay: human embryonic kidney (hek) cells stably expressing human amyloid precursor protein (app) and presenelin i were grown in dmem media (fisher mt10013cv) containing 10% fetal calf serum (fisher #mt135011cv), 0.2 mg/ml g418 (fisher #mt30234cr) and 1× concentration of antibiotic/antimycotic mixture (fisher #mt30004ci). cells were grown in tissue culture flasks and passaged every week at a ratio of 1:30.

Thirty minutes prior to incubation with test compounds, cells were harvested by treating the monolayer with DPBS (Fisher #MT21031CV) containing 3 mM EDTA. Cells were resuspended at a density of 2 million cells/mL in complete growth medium.

Test compounds were solubilized in DMSO at a concentration of 3.3 mM. From this stock solution a dilution series was prepared in complete growth medium of cells. Dilution series were then transferred to 96 well assay plate (Costar #3595) with 100 uL in each well. Cells (100 uL) were added to each well containing test compound. Two controls, one containing only cells (Total) and one containing only growth medium (Background) were also included. Cells were incubated with compounds for 14-16 hours in cell culture incubator.

At the end of 14-16 hour incubation, 100 uL of supernatant was transferred from each well in to a polypropylene 96 well plate. This supernatant was mixed with 100 u L of DPBS (Fisher #MT21031CV) containing 0.5% bovine serum albumin, 0.5% Tween (or an equivalent) 20, 0.25 u g/mL of biotinylated 4G8 (Senetek #240-10), 0.18 u g/mL rabbit anti-A□40 antibody (Biosource #44-3481), 0.045 ug/mL Ruthenium labeled goat anti-rabbit IgG (labeled in-house) and 60 ug/mL of Streptavadin beads (Igen #M280). The mixture was incubated for 4-6 hours at 4° C. on a plate shaker.

At the completion of 4-6 hour incubation, plate was brought to room temperature and the generated A□40 was detected using the Igen M8 analyzer. Raw data was imported into Microsoft Excel software. IC₅₀ values for inhibition of A□40 generation by test compounds were calculated using Excel-Fit.

1. A compound of formula (I): 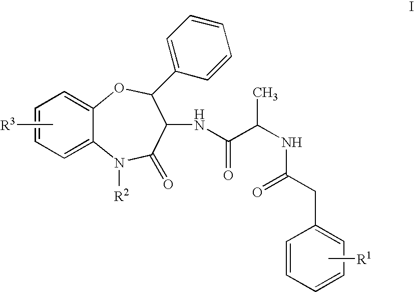
wherein:
R¹ is selected from m-benzoyl, p-benzoyl, or p-azido;
R² is independently selected from H, —CH₃, 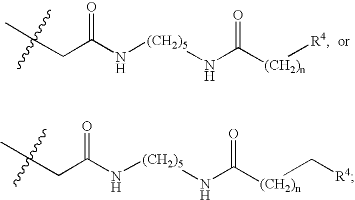
R³ is independently selected from H or 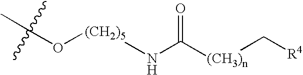
wherein n is 2 or 3; and
R⁴ is selected from:

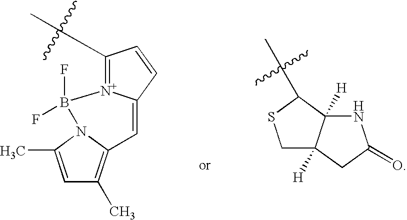

2. A compound according to claim 1 selected from:

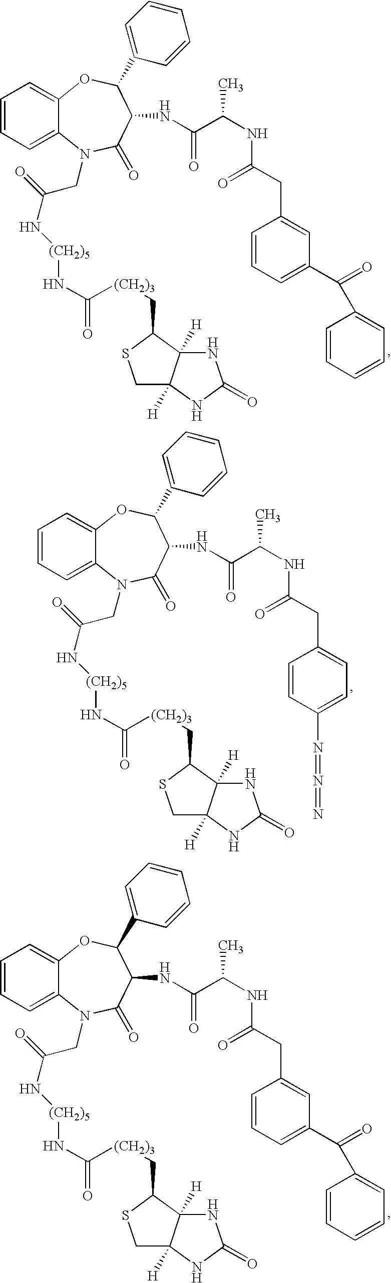

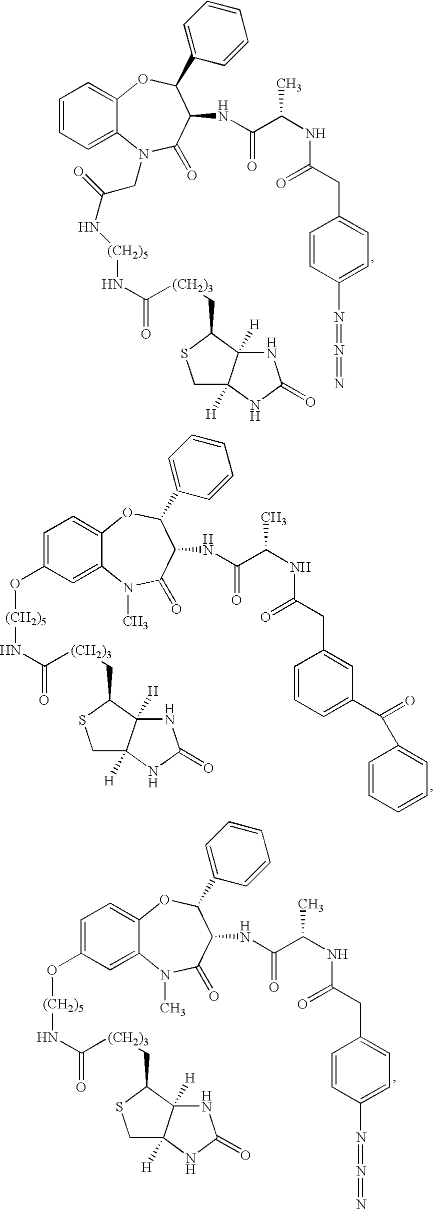

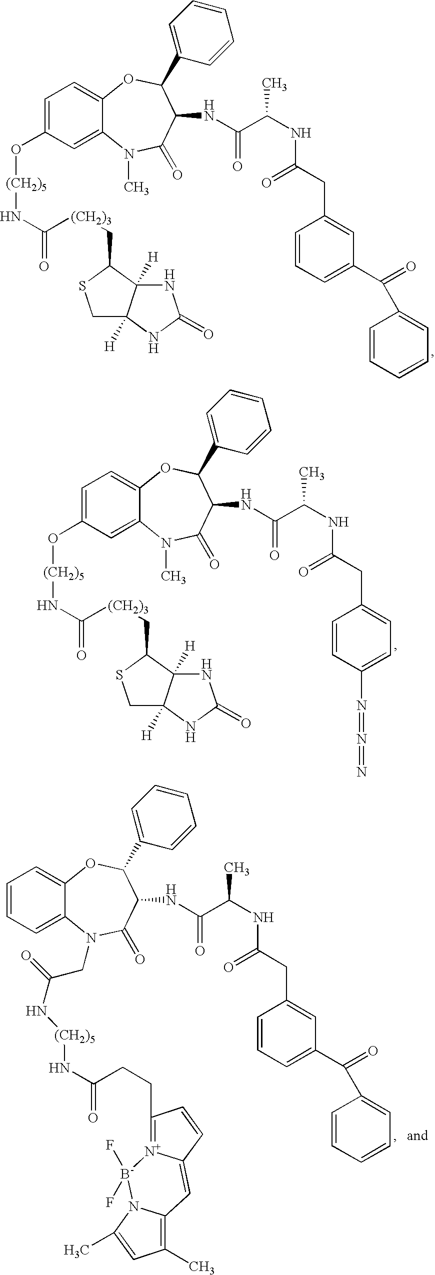

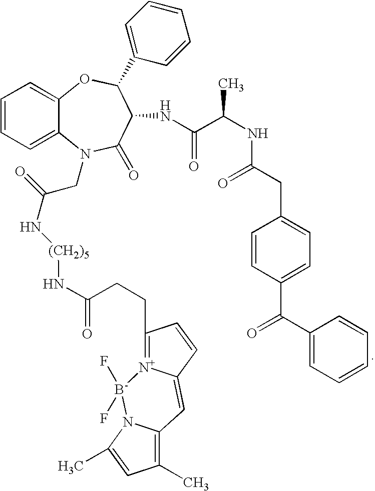

3. A compound according to claim 1 selected from:
N-[5-({[(2R,3R)-3-({N-[(3-benzoylphenyl)acetyl]-L-alanyl}amino)-2-phenyl-3,4-dihydro-1,5-benzoxazepin-5(2H)-yl]acetyl}amino)pentyl]-4-[(3aS,4S,6aR)-2-oxohexahydro-1H-thieno[3,4-d]imidazol-4-yl]butanamide;
N¹-[(2R,3R)-5-(2-oxo-2-{[5-({4-[(3aS,4S,6aR)-2-oxohexahydro-1H-thieno[3,4-d]imidazol-4-yl]butanoyl}amino)pentyl]amino}ethyl)-2-phenyl-2,3,4,5-tetrahydro-1,5-benzoxazepin-3-yl]-N²-[(4-triaza-1,2-dien-2-ium-1-ylphenyl)acetyl]-L-alaninamide;
N-[5-({[(2S,3S)-3-({N-[(3-benzoylphenyl)acetyl]-L-alanyl}amino)-2-phenyl-3,4-dihydro-1,5-benzoxazepin-5(2H)-yl]acetyl}amino)pentyl]-4-[(3aS,4S,6aR)-2-oxohexahydro-1H-thieno[3,4-d]imidazol-4-yl]butanamide;
N¹-[(2S,3S)-5-(2-oxo-2-{[5-({4-[(3aS,4S,6aR)-2-oxohexahydro-1H-thieno[3,4-d]imidazol-4-yl]butanoyl}amino)pentyl]amino}ethyl)-2-phenyl-2,3,4,5-tetrahydro-1,5-benzoxazepin-3-yl]-N²-[(4-triaza-1,2-dien-2-ium-1-ylphenyl)acetyl]-L-alaninamide;
N-(5-{[(2R,3R)-3-({N-[(3-benzoylphenyl)acetyl]-L-alanyl}amino)-5-methyl-2-phenyl-2,3,4,5-tetrahydro-1,5-benzoxazepin-7-yl]oxy}pentyl)-4-[(3aS,4S,6aR)-2-oxohexahydro-1H-thieno[3,4-d]imidazol-4-yl]butanamide;
N¹-((2R,3R)-5-methyl-7-{[5-({4-[(3aS,4S,6aR)-2-oxohexahydro-1H-thieno[3,4-d]imidazol-4-yl]butanoyl}amino)pentyl]oxy}-2-phenyl-2,3,4,5-tetrahydro-1,5-benzoxazepin-3-yl)-N²-[(4-triaza-1,2-dien-2-ium-1-ylphenyl)acetyl]-L-alaninamide;
N-(5-{[(2S,3S)-3-({N-[(3-benzoylphenyl)acetyl]-L-alanyl}amino)-5-methyl-2-phenyl-2,3,4,5-tetrahydro-1,5-benzoxazepin-7-yl]oxy}pentyl)-4-[(3aS,4S,6aR)-2-oxohexahydro-1H-thieno[3,4-d]imidazol-4-yl]butanamide; and
N¹-((2S,3S)-5-methyl-7-{[5-({4-[(3aS,4S,6aR)-2-oxohexahydro-1H-thieno[3,4-d]imidazol-4-yl]butanoyl}amino)pentyl]oxy}-2-phenyl-2,3,4,5-tetrahydro-1,5-benzoxazepin-3-yl)-N²-[(4-triaza-1,2-dien-2-ium-1-ylphenyl)acetyl]-L-alaninamide.

4. A composition comprising a compound according to claim 1.

5. The composition according to claim 4 further comprising dimethylsulfoxide.

6. The composition according to claim 4 further comprising a human cell which comprises amyloid precursor protein and presenelin 1.

7. The composition according to claim 6 further comprising an antibody that binds to human Aβ40 protein.

8. A microtiter plate comprising a compound according to claim 1 and a human cell, wherein the human cell comprises amyloid precursor protein and presenelin 1.

9. The microtiter plate according to claim 8 further comprising an antibody that binds to human Aβ40 protein.
